(19) United States
(12) Patent Application Publication
(10) Pub. No.: US 2026/0107973 A1
(43) Pub. Date: Apr. 23, 2026

(54) AEROSOL-MODIFYING ADDITIVE COMPRISING A SUBSTITUTED 3-(1 METHYLPYRROLIDIN-2-YL)PYRIDINE

(71) Applicant: RAI STRATEGIC HOLDINGS, INC., Winston-Salem, NC (US)

(72) Inventors: Kyle Ford, Pfafftown, NC (US); Brandon S. Darrow, Winston-Salem, NC (US); Michael R. Galloway, Winston-Salem, NC (US); Gary M. Dull, Lewisville, NC (US)

(21) Appl. No.: 19/361,425

(22) Filed: Oct. 17, 2025

Related U.S. Application Data
(60) Provisional application No. 63/708,970, filed on Oct. 18, 2024.
(60) Provisional application No. 63/880,561, filed on Sep. 12, 2025.

(51) Int. Cl. A24D 1/00; A24D 1/02; A24D 1/04; A24D 1/18; A24D 1/20; A24D 3/04; A24D 3/06; A24D 3/14; A24F 40/20; A61K 31/00; A61K 31/4045; A61K 31/465

(52) U.S. Cl. CPC A24D 1/002; A24D 1/027; A24D 1/042; A24D 1/045; A24D 1/18; A24D 1/20; A24D 3/048; A24D 3/061; A24D 3/14; A24F 40/20; A61K 31/4045; A61K 31/465; A61K 31/658

(57) ABSTRACT

Aerosol generating articles adapted for use in an aerosol delivery device are provided. The aerosol generating articles include an aerosol generating material and an aerosol-modifying agent, wherein the aerosol-modifying agent includes a substituted 3-(1-methylpyrrolidin-2-yl)pyridine, a 3-(azetidin-2-yl)pyridine, or a 3-(azetidin-2-ylmethoxy)pyridine. The disclosure further provides devices incorporating such aerosol generating articles.

### CROSS-REFERENCE TO RELATED APPLICATIONS

This application claims priority to U.S. Provisional Application No. 63/708,970, filed on Oct. 18, 2024, and claims priority to U.S. Provisional Application No. 63/880,561, filed on Sep. 12, 2025, each of which is incorporated herein by reference in its entirety and for all purposes.

### FIELD OF THE DISCLOSURE

The present disclosure relates to an aerosol generating assembly and components thereof.

### BACKGROUND

Aerosol provision systems, and in particular, non-combustible aerosol provision systems have been proposed through the years as improvements upon, or alternatives to, smoking products that require combusting tobacco for use. These systems are generally designed to deliver at least one substance to a user, such as to satisfy a particular “consumer moment.” To this end, the substance may include constituents that impart a physiological effect on the user, a sensorial effect on the user, or both. The substance may be generally present in an aerosol-generating material that may contain one or more constituents of a range of constituents, such as active substances, flavors, aerosol former materials and other functional materials like fillers.

Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs). Many of these products take the form of a system including a device and a consumable, and it is the consumable that includes the material from which the substance to be delivered originates. Typically, the device is reusable, and the consumable is single use (although some consumables are refillable). Therefore, in many cases, the consumable is sold separately from the device, and often in a multipack. Moreover, subsystems and some individual components of devices or consumables may be sourced from specialist manufacturers. Alternatively, aerosol provision systems may be disposable, such that once the vapor forming material is depleted, the entire device is discarded.

### BRIEF SUMMARY

The present disclosure is generally directed to an aerosol generating article comprising an aerosol generating material and an aerosol-modifying agent. The aerosol-modifying agent comprises a substituted 3-(1-methylpyrrolidin-2-yl)pyridine, a 3-(azetidin-2-yl)pyridine, or a 3-(azetidin-2-ylmethoxy)pyridine. The present disclosure includes, without limitation, the following embodiments.

Embodiment 1: An aerosol generating article comprising an aerosol generating material and an aerosol-modifying agent, wherein the aerosol-modifying agent comprises a compound having a structure according to Formula I, Formula II, or Formula III:

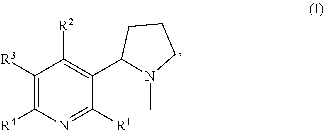

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

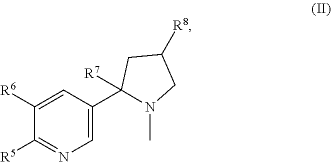

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C1-C3 alkyl; and
   at least one of R⁷ and R⁸ is not hydrogen; or

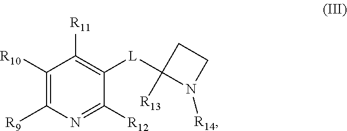

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

Embodiment 2: The aerosol generating article of Embodiment 1, wherein the aerosol-modifying agent comprises a compound having a structure according to Formula I, and wherein R¹, R², and R³ are each H.

Embodiment 3: The aerosol generating article of Embodiment 1 or 2, wherein the aerosol-modifying agent comprises a compound having a structure according to Formula I, and wherein R⁴ is optionally substituted C₁-C₆ alkyl, F, Cl, Br, OCH₃, OEt, or CN.

Embodiment 4: The aerosol generating article of Embodiment 3, wherein R⁴ is C₁-C₃ alkyl, such as wherein R⁴ is CH₃.

Embodiment 5: The aerosol generating article of Embodiment 1, wherein the aerosol-modifying agent comprises a compound having a structure according to Formula II, and wherein R⁵ and R⁶ are H; R⁷ is CH₃; and R⁸ is H.

Embodiment 6: The aerosol generating article of Embodiment 1, wherein the aerosol-modifying agent comprises a compound having a structure according to Formula II, and wherein R⁵, R⁶ and R⁷ are H, and R⁸ is CH₃.

Embodiment 7: The aerosol generating article of any one of Embodiments 1 to 6, wherein the aerosol-modifying agent further comprises an active agent, a flavorant, or a combination thereof.

Embodiment 8: The aerosol generating article of any one of Embodiments 1 to 7, wherein the active agent comprises a nicotine component, a cannabinoid, a terpene, caffeine, an amino acid, a vitamin, melatonin, a botanical extract, or a combination thereof.

Embodiment 9: The aerosol generating article of any one of Embodiments 1 to 8, further comprising a filter, a cooling element, or both a filter and a cooling element, optionally wherein the aerosol-modifying agent is present in the filter and/or the cooling element.

Embodiment 10: The aerosol generating article of any one of Embodiments 1 to 9, further comprising a mouthpiece having an upstream end and a downstream end, the mouthpiece comprising a hollow tubular element formed from filamentary tow at the downstream end of the mouthpiece.

Embodiment 11: The aerosol generating article of Embodiment 10, wherein the mouthpiece comprises a body of material in the form of a cylinder having a longitudinal axis.

Embodiment 12: The aerosol generating article of any one of Embodiments 1 to 11, wherein the aerosol-modifying agent is retained within an aerosol-modifying agent release component.

Embodiment 13: The aerosol generating article of Embodiment 12, wherein the aerosol-modifying agent release component is in the form of a coating, a capsule, a thread, a bead, or a paper.

Embodiment 14: The aerosol generating article of any one of Embodiments 1 to 13, wherein the aerosol-modifying agent release component is present in the form of one or more capsules comprising a shell encapsulating the aerosol-modifying agent, and optionally wherein the mouthpiece comprises a body of material in the form of a cylinder having a longitudinal axis, and wherein the one or more capsules are embedded within the body of material such that the one or more capsules are surrounded on all sides by the material forming the body of material.

Embodiment 15: The aerosol generating article of Embodiment 14, wherein the shell comprises a barrier material comprising a gelling agent, a bulking agent, a filler, a buffer, a coloring agent, a plasticizer, or a combination thereof.

Embodiment 16: The aerosol generating article of Embodiment 14, wherein the one or more capsules are spherical and have a diameter in a range from about 0.4 mm to about 3 mm.

Embodiment 17: The aerosol generating article of any one of Embodiments 1 to 16, comprising a wrapper which at least partially surrounds the article, and wherein the aerosol-modifying agent is absorbed or adsorbed in or on the wrapping material.

Embodiment 18: The aerosol generating article of Embodiment 17, wherein the wrapper comprises ventilation apertures.

Embodiment 19: The aerosol generating article of any one of Embodiments 1 to 18, wherein the aerosol generating material is in the form of a rod (e.g., a substantially cylindrical rod), optionally wherein the rod has a length in a range from about 10 mm to about 100 mm.

Embodiment 20: The aerosol generating article of Embodiment 19, wherein the rod has a cavity therein, and wherein an aerosol-modifying agent release component is present within the cavity in the form of one or more capsules comprising a shell encapsulating the aerosol-modifying agent.

Embodiment 21: The aerosol generating article of any one of Embodiments 1 to 20, wherein the aerosol generating material is a solid and optionally comprises a botanical material.

Embodiment 22: The aerosol generating article of any one of Embodiments 1 to 21, which is substantially free of a nicotine component.

Embodiment 23: The aerosol generating article of any one of Embodiments 1 to 21, which is substantially free of tobacco material.

Embodiment 24: An aerosol generating assembly comprising:
(i) an aerosol generating device comprising a coil; and
(ii) the aerosol generating article of any one of Embodiments 1-23, wherein the aerosol generating article and the aerosol generating device are arranged with respect to each other such that the aerosol generating material is heatable by the aerosol generating device.

Embodiment 25: The aerosol generating assembly of Embodiment 24, wherein the aerosol generating material is in the form of a rod having a cavity therein, and wherein an aerosol-modifying agent release component is present within the cavity in the form of one or more capsules comprising a shell encapsulating the aerosol-modifying agent, and wherein the aerosol generating device comprises an aerosol generator adapted to enter the cavity in the rod and pierce the one or more capsules to release the aerosol-modifying agent.

Embodiment 26: The aerosol generating article of Embodiment 1, wherein the aerosol-modifying agent comprises a compound having a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 27: The aerosol generating article of Embodiment 1, wherein the aerosol-modifying agent comprises a compound having a structure according to Formula III, optionally wherein R⁹ is H or CH₃; and R¹⁰, R¹¹, and R¹² are each H.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 shows a front view of an aerosol generating device according to a non-limiting embodiment of the disclosure.

FIG. 2 shows a front view of the aerosol generating device of FIG. 1 with an outer cover removed.

FIG. 3A shows a cross-sectional view of a heating assembly within an aerosol generating device according to a non-limiting embodiment of the disclosure.

FIG. 3B shows a close-up view of a portion of the heating assembly of FIG. 3A.

FIG. 4A shows a partially cut-away section view of an aerosol generating article according to a non-limiting embodiment of the disclosure.

FIG. 4B shows a perspective view of the aerosol generating article of FIG. 4A.

FIG. 5 shows a side-on cross-sectional view of an article according to a non-limiting embodiment of the disclosure for use with a non-combustible aerosol provision device, the article including a mouthpiece.

FIG. 6A shows a side-on cross-sectional view of a further article according to a non-limiting embodiment of the disclosure for use with a non-combustible aerosol provision device, the article including a capsule-containing mouthpiece.

FIG. 6B shows a cross sectional view of the capsule-containing mouthpiece shown in FIG. 6A.

FIG. 7 is a cross-sectional view of a further article according to a non-limiting embodiment of the disclosure for use with a non-combustible aerosol provision device, the article including a capsule.

FIG. 8 is a cross-sectional view of a non-combustible aerosol provision device according to a non-limiting embodiment of the disclosure for use with the article of FIG. 7.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated. The terms ‘upstream’ and ‘downstream’ used herein are relative terms defined in relation to the direction of mainstream aerosol drawn though an article or device in use.

The terms ‘upstream’ and ‘downstream’ used herein are relative terms defined in relation to the direction of mainstream aerosol drawn though an article or device in use.

Unless otherwise defined herein, by “substantially free” it is meant that the noted component (e.g., nicotine or a tobacco material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the total weight of the aerosol generating material. In some embodiments, the aerosol generating material is completely free of the noted component (i.e., having 0% or as having an amount below the limit of detection).

The present disclosure is generally directed to an aerosol generating article comprising an aerosol generating material and an aerosol-modifying agent, and to an aerosol generating assembly comprising an aerosol generating device and the aerosol generating article. Each of the articles, devices, materials, and components of each thereof are further described herein below. In some embodiments, it is advantageous to keep the aerosol-modifying agent separate from the aerosol generating material until the use of the aerosol generating device by the user. For example, in some embodiments, segregation of the aerosol-modifying agent and the aerosol generating material may extend shelf-life.

### Aerosol Generating Material

In one aspect of the disclosure is provided an aerosol generating material. As used herein, the term “aerosol generating material” includes materials that provide volatilized components upon heating, typically in the form of an aerosol. Aerosol generating materials may also be referred to as “smokable materials”, “aerosolizable materials”, or “aerosol generating substrates.”

Aerosol-generating material is a material that is capable of generating aerosol, for example when heated, irradiated or energized in any other way. Aerosol-generating material may, for example, be in the form of a solid, liquid or semi-solid (such as a gel) which may or may not contain an active substance and/or flavorants. In some examples, the aerosol-generating material is substantially free of botanical material. In particular, in some examples, the aerosol-generating material is substantially free of tobacco material.

The aerosol-generating material may comprise, for example, a base material, one or more active ingredients and/or flavors, one or more aerosol-former materials, and optionally one or more other functional materials. The form of the aerosol-generating material may vary, and can include films, sheets, gathered paper, shredded material, extruded material, and the like.

The aerosol-generating material may comprise a binder, such as a gelling agent, and an aerosol former. Optionally, a substance to be delivered and/or filler may also be present. Optionally, a solvent, such as water, is also present and one or more other components of the aerosol-generating material may or may not be soluble in the solvent.

The aerosol-generating material may comprise or be in the form of an aerosol-generating film. The aerosol-generating film may comprise a binder, such as a gelling agent, and an aerosol former. Optionally, a substance to be delivered and/or filler may also be present.

The aerosol-generating film may have a thickness of about 0.015 mm to about 1 mm. For example, the thickness may be in the range of about 0.05 mm, 0.1 mm or 0.15 mm to about 0.5 mm or 0.3 mm.

The aerosol-generating film may be continuous. For example, the film may comprise or be a continuous sheet of material. The sheet may be in the form of a wrapper, it may be gathered to form a gathered sheet or it may be shredded to form a shredded sheet. The shredded sheet may comprise one or more strands or strips of aerosol-generating material.

The aerosol-generating film may be discontinuous. For example, the aerosol-generating film may comprise one or more discrete portions or regions of aerosol-generating material, such as dots, stripes or lines, which may be supported on a support. In such examples, the support may be planar or non-planar.

The aerosol-generating film may be formed by combining a binder, such as a gelling agent, with a solvent, such as water, an aerosol-former and one or more other components, such as one or more substances to be delivered, to form a slurry and then heating the slurry to volatilize at least some of the solvent to form the aerosol-generating film. The slurry may be heated to remove at least about 60 wt %, 70 wt %, 80 wt %, 85 wt % or 90 wt % of the solvent.

The aerosol-generating material may comprise or be an “amorphous solid”. In some examples, the aerosol-generating materiel comprises an aerosol-generating film that is an amorphous solid. The amorphous solid may be a “monolithic solid”. The amorphous solid may be substantially non-fibrous. In some examples, the amorphous solid may be a dried gel. The amorphous solid is a solid material that may retain some fluid, such as liquid, within it. In some examples, the amorphous solid may, for example, comprise from about 50 wt %, 60 wt % or 70 wt % of amorphous solid, to about 90 wt %, 95 wt % or 100 wt % of amorphous solid.

The aerosol-generating material may comprise a base material that serves as a carrier for other components of the aerosol-generating material, such as active ingredients, flavorants, and aerosol forming materials. Aerosol generating materials may comprise a combination or a blend of materials.

In some embodiments, the aerosol generating material comprises shredded paper as a base material. In some embodiments, the base material comprises a woven or nonwoven material such as lyocell, viscose, fibrous materials such as cellulose fibers, or a combination thereof. In some embodiments, the base material comprises a botanical material, such as any of the botanical materials as described herein with respect to active ingredient botanical materials (e.g., rooibos).

In some embodiments, the aerosol generating material comprises a tobacco material. As used herein, the term “tobacco material” refers to any material comprising tobacco or derivatives thereof. The term “tobacco material” may include one or more of tobacco, tobacco derivatives, expanded tobacco, reconstituted tobacco or tobacco substitutes. The tobacco material may comprise one or more of ground tobacco, tobacco fiber, cut tobacco, extruded tobacco, tobacco stem, reconstituted tobacco and/or tobacco extract. The tobacco used to produce tobacco material may be any suitable tobacco, such as single grades or blends, cut rag or whole leaf, including Virginia and/or Burley and/or Oriental. It may also be tobacco particle ‘fines’ or dust, expanded tobacco, stems, expanded stems, and other processed stem materials, such as cut rolled stems. The tobacco material may be a ground tobacco or a reconstituted tobacco material. The reconstituted tobacco material may comprise tobacco fibers, and may be formed by casting, a Fourdrinier-based paper making-type approach with back addition of tobacco extract, or by extrusion. In some embodiments, the aerosol generating material comprises a reconstituted tobacco material. In some embodiments, the aerosol generating material comprises paper reconstituted tobacco. The aerosol generating material may comprise or further comprise leaf tobacco, extruded tobacco, and/or bandcast tobacco.

In some embodiments, the aerosol generating material comprises paper reconstituted tobacco. Paper reconstituted tobacco refers to tobacco material formed by a process in which tobacco feedstock is extracted with a solvent to afford an extract of solubles and a residue comprising fibrous material, and then the extract (usually after concentration, and optionally after further processing) is recombined with fibrous material from the residue (usually after refining of the fibrous material, and optionally with the addition of a portion of non-tobacco fibers) by deposition of the extract onto the fibrous material. The process of recombination resembles the process for making paper.

In some embodiments, the aerosol generating material is substantially free of tobacco material.

### Filler Component

In some embodiments, the aerosol generating material comprises a filler. As used herein, the term “filler” may refer to one or more inorganic filler materials, such as calcium carbonate, perlite, vermiculite, diatomaceous earth, colloidal silica, magnesium oxide, magnesium sulphate, magnesium carbonate, and suitable inorganic sorbents, such as molecular sieves. Alternatively, the term filler may refer to one or more organic filler materials such as wood pulp, cellulose and cellulose derivatives. The filler may comprise organic and inorganic filler materials. Generally, the filler is a non-tobacco component, that is, a component that does not include ingredients originating from tobacco. The filler may be a non-tobacco fiber such as wood fiber or pulp or wheat fiber. The filler may also be an inorganic material such as chalk, perlite, vermiculite, diatomaceous earth, colloidal silica, magnesium oxide, magnesium sulphate, magnesium carbonate. The filler may also be a non-tobacco cast material or a non-tobacco extruded material. The filler may be present in an amount of 0 to 20% by weight, or in an amount of from 1 to 10% by weight, based on the total weight of the aerosol generating material.

### Binder

In some embodiments, the aerosol generating material comprises a binder. As used herein, the term “binder” may refer to alginates, celluloses or modified celluloses, starches or modified starches, or natural gums. Suitable binders include, but are not limited to, alginate salts comprising any suitable cation; celluloses or modified celluloses, such as hydroxypropyl cellulose and carboxymethylcellulose; starches or modified starches; polysaccharides such as pectin salts comprising any suitable cation, such as sodium, potassium, calcium or magnesium pectate; xanthan gum, guar gum, and any other suitable natural gums; and mixtures thereof. In some embodiments, the binder comprises, substantially consists of or consists of one or more alginate salts selected from sodium alginate, calcium alginate, potassium alginate or ammonium alginate.

### Aerosol Forming Material

In some embodiments, the aerosol generating material comprises an aerosol forming material. In this context, an “aerosol forming material” is an agent that promotes the generation of an aerosol. An aerosol forming material may promote the generation of an aerosol by promoting an initial vaporization and/or the condensation of a gas to an inhalable solid and/or liquid aerosol. In some embodiments, an aerosol forming material may improve the delivery of flavor from the aerosol generating material.

The aerosol forming material may be included in any component of the aerosol generating material, for example, the base material, filler, or combinations thereof. The total amount of the aerosol forming material in the aerosol generating material may vary.

In general, any suitable aerosol forming material or agents may be included in the aerosol generating material of the disclosure. The particular choice of aerosol former(s) may depend on factors such as the method of aerosol formation, the appearance and volume of the aerosol, the desired density of the aerosol, and the like. Suitable aerosol forming materials include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate. In some embodiments, the aerosol forming material may be glycerol, propylene glycol, or a mixture of glycerol and propylene glycol.

In some embodiments, the aerosol forming material may include one or more polyhydric alcohols. Examples of polyhydric alcohols include glycerol, propylene glycol, and other glycols such as 1,3-propanediol, diethylene glycol, triethylene glycol, and polyethylene glycols (e.g., PEG molecules with weight average molecular weight range of about 200 to about 2,000 Da). In some embodiments, the aerosol former comprises one or more polyhydric alcohols. In some embodiments, the one or more polyhydric alcohols are selected from the group consisting of glycerol, propylene glycol, 1,3-propanediol, diethylene glycol, triethylene glycol, a polyethylene glycol, and combinations thereof. In some embodiments, the polyhydric alcohol is selected from the group consisting of glycerol, propylene glycol, and mixtures thereof. In some embodiments, the aerosol forming material is substantially free of propylene glycol. In some embodiments, the aerosol forming material is substantially free of glycerin.

In some embodiments, the aerosol forming material comprises a combination of propylene glycol and glycerol. The relative amounts of propylene glycol and glycerol may vary and may be expressed as a weight ratio. In some embodiments, the forming material comprises propylene glycol and glycerol in a ratio by weight from about 1:3 to about 3:1, such as about 1:3, about 1:2, about 1:1, about 2:1, or about 3:1.

In some embodiments, the aerosol forming material further comprises one or more polysorbates, one or more sorbitan esters, one or more fatty acids, one or more fatty acid esters, triacetin, one or more waxes, one or more sugar alcohols, or combinations thereof. Examples of polysorbates include, but are not limited to, Polysorbate 60 (polyoxyethylene (20) sorbitan monostearate, Tween® 60) and Polysorbate 80 (polyoxyethylene (20) sorbitan monooleate; Tween® 80). Examples of sorbitan esters include, but are not limited to, sorbitan monolaurate, sorbitan monostearate (Span® 60), sorbitan monooleate (Span® 20), and sorbitan tristearate (Span® 65). Examples of fatty acids include, but are not limited to, butyric, propionic, valeric, oleic, linoleic, stearic, myristic, and palmitic acids. Examples of fatty acid esters include, but are not limited to, alkyl esters, monoglycerides, diglycerides, and triglycerides. Examples of monoglycerides include monolaurin and glycerol monostearate. Examples of triglycerides include triolein, tripalmitin, tristearate, glycerol tributyrate, and glycerol trihexanoate. Examples of waxes include, but are not limited to, carnauba, beeswax, candellila. Examples of sugar alcohols include, but are not limited to, sorbitol, erythritol, mannitol, maltitol, isomalt, and xylitol. In some embodiments, the aerosol former further comprises erythritol, meso-erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, or combinations thereof.

### Active Ingredient

In some embodiments, the aerosol generating material comprises an active ingredient. The active ingredient may be a physiologically active material, which is a material intended to achieve or enhance a physiological response. The active ingredient may for example be selected from nutraceuticals, nootropics, psychoactives. The active ingredient may be naturally occurring or synthetically obtained. The active ingredient may comprise for example nicotine, caffeine, taurine, theanine, vitamins such as B6 or B12 or C, melatonin, cannabinoids, or constituents, derivatives, or combinations thereof. The active ingredient may comprise one or more constituents, derivatives or extracts of cannabis or another botanical (other than tobacco).

### Botanical Material

In some embodiments, the active ingredient comprises one or more non-tobacco botanicals. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, or other treatment processes capable of altering the chemical nature of the material). For the purposes of the present disclosure, a “botanical material” includes but is not limited to “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). The botanical materials used in the present disclosure may comprise, without limitation, any of the compounds and sources set forth herein, including mixtures thereof.

Non-limiting examples of botanical materials include without limitation acai berry, alfalfa, allspice, annatto seed, apricot oil, ashwagandha, bacopa monniera, baobab, basil, bee balm, beet root, wild bergamot, black pepper, blueberries, borage seed oil, bugleweed, cacao, calamus root, catnip, catuaba, cayenne pepper, Centella asiatica, chaga mushroom, Chai-hu, chamomile, cherry blossom, chervil, chlorophyll, cinnamon, dark chocolate, citrus, cocoa, comfrey leaf and root, gingko biloba, ginseng, goji berries, grape seed, green tea, black tea, black cohosh, cayenne, chamomile, cloves, cocoa powder, cordyceps, cranberry, curcumin, damiana, dandelion, Dorstenia arifolia, Dorstenia odorata, echinacea, eucalyptus, fennel, feverfew, Galphimia glauca, garlic, ginger, ginseng (e.g., Panax ginseng), goldenseal, green tea, grapefruit, Griffonia simplicifolia, guarana, gutu kola, hawthorn, hemp, hibiscus flower, honeybush, hops, jasmine, jiaogulan, Kaempferia parviflora (Thai ginseng), kava, lavender, lemon balm, lemongrass, licorice, Lion's mane, lutein, maca, matcha, Nardostachys chinensis, marjoram, milk thistle, mints (menthe), oolong tea, orange, oregano, papaya, pennyroyal, peppermint, potato peel, primrose, quercetin, red clover, resveratrol, Rhizoma gastrodiae, Rhodiola, Aspalathus linearis (rooibos; red or green), rose essential oil, rosehip, rosemary, sage, clary sage, savory, saw palmetto, Sceletium tortuosum, Schisandra, silybum marianum, Skullcap, spearmint, Spikenard, spirulina, slippery elm bark, sorghum bran hi-tannin, sorghum grain hi-tannin, Saint John's Wort, sumac bran, terpenes, thyme, tisanes, turmeric, Turnera aphrodisiaca, uva ursi, valerian, Viola odorata, white mulberry, wild yam root, wintergreen, withania somnifera, yacon root, yellow dock, yerba mate, and yerba santa.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the aerosol generating material.

### Cannabinoids

In some embodiments, the active ingredient comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example 48- and 49-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is 48-CBD. In some embodiments, the CBD is 49-CBD.

In some embodiments, the cannabinoid (e.g., CBD) is added to the aerosol generating material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol generating material is no greater than about 1% by weight of the aerosol generating material, such as no greater than about 0.5% by weight of the aerosol generating material such as no greater than about 0.1% by weight of the aerosol generating material such as no greater than about 0.01% by weight of the aerosol generating material.

The choice of cannabinoid and the particular percentages thereof which may be present within the aerosol generating material will vary depending upon the desired characteristics of the material.

In some embodiments, the cannabinoid (such as CBD) is present in the aerosol generating material in a concentration of at least about 0.001% by weight of the aerosol generating material, such as in a range from about 0.001% to about 2% by weight of the aerosol generating material. In some embodiments, the cannabinoid (such as CBD) is present in the aerosol generating material in a concentration of from about 0.1% to about 1.5% by weight, based on the total weight of the aerosol generating material. In some embodiments, the cannabinoid (such as CBD) is present in a concentration from about 0.4% to about 1.5% by weight, based on the total weight of the aerosol generating material.

Alternatively, or in addition to a cannabinoid, the active ingredient may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Active ingredients suitable for use in the aerosol generating material can also be classified as terpenes, many of which are associated with biological effects, such as calming effects. Terpenes are understood to have the general formula of (C₅H₈), and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active ingredient, as an aerosol forming material, or as a flavorant. The amount of terpene and/or cannabinoid present may vary accordingly based on their intended purpose.

### Nicotine

In some embodiments, the aerosol generating material comprises nicotine. In some embodiments, the aerosol generating material, which may comprise a tobacco material, suitably a reconstituted tobacco material, may have a nicotine content of between about 5 mg/g and 15 mg/g (dry weight basis), suitably between about 7 mg/g and 12 mg/g. In some embodiments, the aerosol generating material comprises at least about 1.5 mg of nicotine, suitably at least about 1.7 mg, 1.8 mg or 1.9 mg of nicotine. In some embodiments, the aerosol generating material is substantially free of nicotine.

In some embodiments, the aerosol generating material of the disclosure can be completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in the substituted 3-(1-methylpyrrolidin-2-yl)pyridine. For example, some embodiments can have less than 0.01% by weight of nicotine, or less than 0.001% by weight of nicotine, or less than 0.0001%, or even 0% by weight of nicotine, calculated as the free base and based on the total weight of the material. In some embodiments, the material is completely free of (R)-, (S)-, and (R\S)-3-(1-methylpyrrolidin-2-yl)pyridine (e.g., having 0% by weight of nicotine, including racemic nicotine and nicotine enantiomers, calculated as the free base and based on the total weight of the aerosol generating material).

### Flavoring Agent

In some embodiments, the aerosol generating material as described herein comprises a flavoring agent. As used herein, a “flavoring agent” or “flavorant” is any flavorful or aromatic substance capable of altering the sensory characteristics associated with the aerosol generating material or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the aerosol generating material comprises a sensate which provides to the user of such aerosol generating material a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA 4693), WS-116 (FEMA 4603), N-ethyl-2,2-diisopropylbutanamide, isopulegol, menthyloxy propane diol, 3-(1-menthoxy) propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy) ethan-1-ol, 3-(1-menthoxy) propan-1-ol, 3-(1-menthoxy) butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy) ethoxy]ethanol, (1R,2R,4R)-1-(2-Hydroxy-4-methylcyclohexyl) ethenone, 2-(p-tolyloxy)-N-(1H-pyrazol-5-yl)-N-((thiophen-2-yl)methyl) acetamide, menthol methyl ether, menthyl pyrrolidone carboxylate, 2,5-dimethyl-4-(1-pyrrolidinyl)-3 (2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)—N-(4-(cyanomethyl)phenyl) menthylcarboxamide (Evercool™ 180), (1R,2S,5R)—N-(2-(pyridin-2-yl)ethyl) menthylcarboxamide (Evercool™ 190), or a combination thereof.

In some embodiments, the aerosol generating material does not comprise a flavoring agent and comprises only a cooling agent to provide the desired user experience. In some embodiments, the cooling agent is WS-3.

In some embodiments, the aerosol generating material comprises a modulator or sensate which provides to the user of such material a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the aerosol generating material can vary, but is typically up to about 10% by weight, and certain embodiments have a flavoring agent content of at least about 0.1% by weight, such as about 0.5 to about 10%, about 1 to about 5%, or about 2 to about 4% weight, based on the total weight of the aerosol generating material.

### Form of Aerosol Generating Material

The form of an aerosol generating material may vary. For example, an aerosol generating material of the present disclosure may be in the form of a solid, a liquid, a gel, a wax, or the like. In some embodiments, the aerosol generating material is a solid. In some embodiments, the aerosol generating material is a solid and optionally comprises a botanical material. In some embodiments, the aerosol generating material is a solid and comprises a botanical material. In some embodiments, the aerosol generating material is a solid and comprises a tobacco material.

In some embodiments, the aerosol generating material is in the form of a substantially cylindrical rod having a length in a range from about 10 mm to about 100 mm. In some embodiments, the cylindrical rod of aerosol generating material is between about 34 mm and 50 mm in length, suitably between about 38 mm and 46 mm in length, suitably about 42 mm in length. The cylindrical body of aerosol generating material may have a diameter of about 5.0 mm to 6.0 mm, suitably about 5.25 mm to 5.45 mm, suitably about 5.35 mm to 5.40 mm, or suitably about 5.39 mm.

### Aerosol-Modifying Agent

An aerosol-modifying agent is a substance, typically located downstream of the aerosol generation area, that is configured to modify the aerosol generated, for example by changing the taste, flavor, acidity or another characteristic of the aerosol. The aerosol-modifying agent may be provided in an aerosol-modifying agent release component, that is operable to selectively release the aerosol-modifying agent.

The aerosol-modifying agent may, for example, be an additive or a sorbent. The aerosol-modifying agent may, for example, comprise one or more of a flavorant, a colorant, water, and a carbon adsorbent. The aerosol-modifying agent may, for example, be a solid, a liquid, or a gel. The aerosol-modifying agent may be in powder, thread or granule form. The aerosol-modifying agent may be free from filtration material.

The form of the aerosol-modifying agent release component can vary. In some embodiments, the aerosol-modifying agent release component is a coating, a capsule, a thread, a bead, or a paper, such as a plug wrap, a tipping paper or a cigarette paper.

In some embodiments, the aerosol-modifying agent comprises a substituted 3-(1-methylpyrrolidin-2-yl)pyridine, a 3-(azetidin-2-yl)pyridine, or a 3-(azetidin-2-ylmethoxy)pyridine as described herein.

In some embodiments, the aerosol-modifying agent comprises a substituted 3-(1-methylpyrrolidin-2-yl)pyridine. As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring.

In some embodiments, the substituted 3-(1-methylpyrrolidin-2-yl)pyridine has a structure according to Formula I:

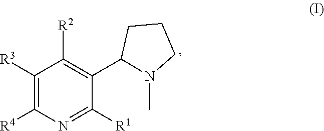

wherein:
   R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34 (1): 31-40.

In some embodiments, R¹, R², and R⁴ are each H, and R³ is a non-hydrogen substituent. In some embodiments, R³ is optionally substituted C₁-C₆ alkyl, F, Cl, Br, OMe, OEt, or CN. In some embodiments, R³ is Me or Et. n some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the aerosol-modifying agent of the disclosure comprises a substituted 3-(1-methylpyrrolidin-2-yl)pyridine having a structure according to Formula II:

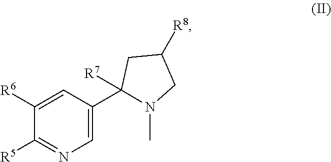

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C1-C3 alkyl; and at least one of R⁷ and R⁸ is not hydrogen.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34 (1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

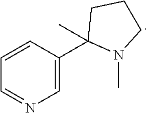

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49 (1), 161-166; Wang et al., Drug Dev Res 1998, 45(1), 10-16; Secor et al., Tetrahedron Lett. (1981), 22 (33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

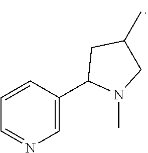

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

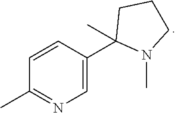

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

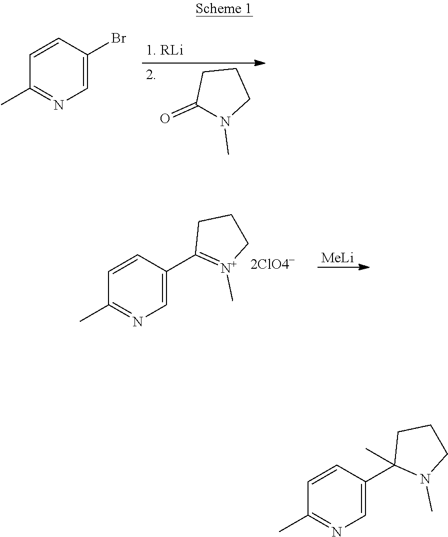

In some embodiments, R⁵ is H or CH₃, R⁶ is H, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from

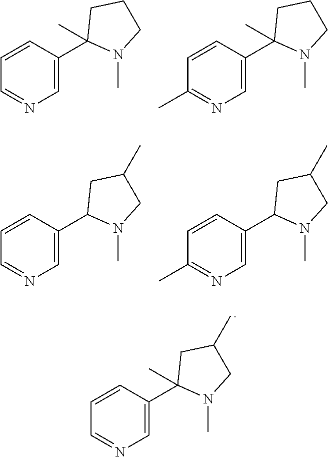

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from

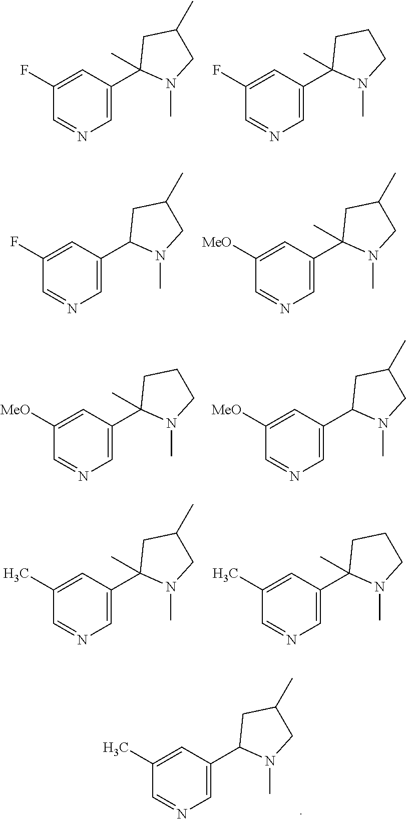

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the aerosol-modifying agent comprises unequal amounts of (R)- and (S)-enantiomer (i.e., is enriched in either the (R)- or (S)-enantiomer. In some embodiments, the aerosol-modifying agent predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the aerosol-modifying agent predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the aerosol-modifying agent comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

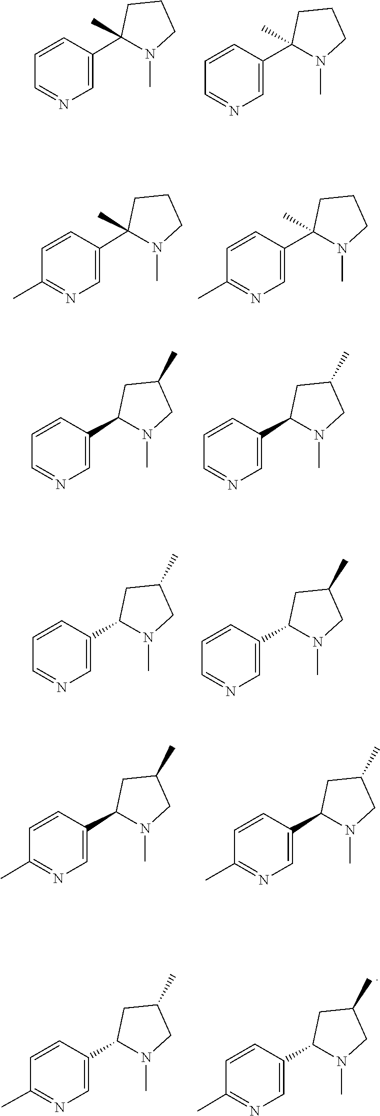

In some embodiments, the substituted 3-(1-methylpyrrolidin-2-yl)pyridine bearing one or more substituents on the pyrrolidine ring is non-racemic, and has a structure selected from:

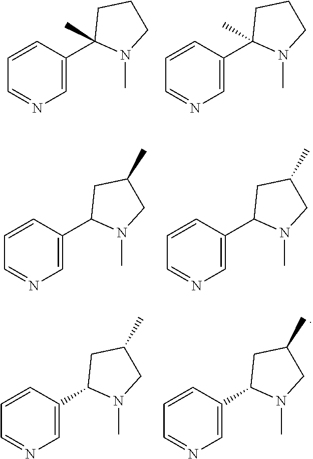

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31 (11), 1996, 875-888; U.S. Pat. No. 9,440,948; Wang et al., Drug Dev Res 1998, 45 (1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45 (1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The quantity of substituted 3-(1-methylpyrrolidin-2-yl)pyridine (e.g., 2-methyl-5-(1-methylpyrrolidin-2-yl)pyridine) present in the aerosol generating material may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the article, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the article. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the article. In some embodiments, the article comprises 2-methyl-5-(1-methylpyrrolidin-2-yl)pyridine in an amount from about 0.01 to about 1% by weight, such as from about 0.1 to about 0.75% by weight, based on the total weight of the article.

The substituted 3-(1-methylpyrrolidin-2-yl)pyridine (e.g., 2-methyl-5-(1-methylpyrrolidin-2-yl)pyridine) may be present in the article (e.g., in the aerosol generating material) as the free base, as a salt with a suitable acid, in the form of an ion pair with an organic acid, or a combination thereof. Each of these forms is described further herein below.

In some embodiments, the aerosol-modifying agent of the disclosure comprises an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

In some embodiments, the 3-(azetidin-2-yl)pyridine or 3-(azetidin-2-ylmethoxy)pyridine has a structure according to Formula III:

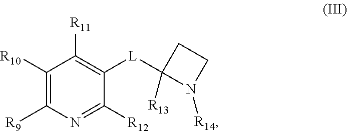

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-yl)pyridine is 3-(azetidin-2-yl)pyridine, and has a structure:

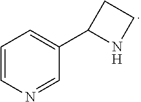

The compound 3-(azetidin-2-yl)pyridine is known in the literature. The synthesis of this compound has been reported in JOC 1979, 44 (18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl)pyridine is 3-(1-methylazetidin-2-yl)pyridine, having the structure:

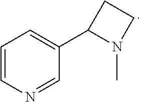

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl)pyridine has a structure selected from the group consisting of:

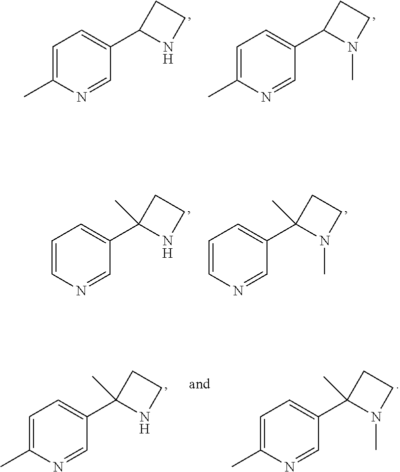

Such compounds are either known, or may be readily prepared according to adaptations of methods utilized for preparation of related 3-(azetidin-2-yl)pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the aerosol-modifying agent comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

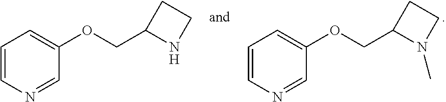

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

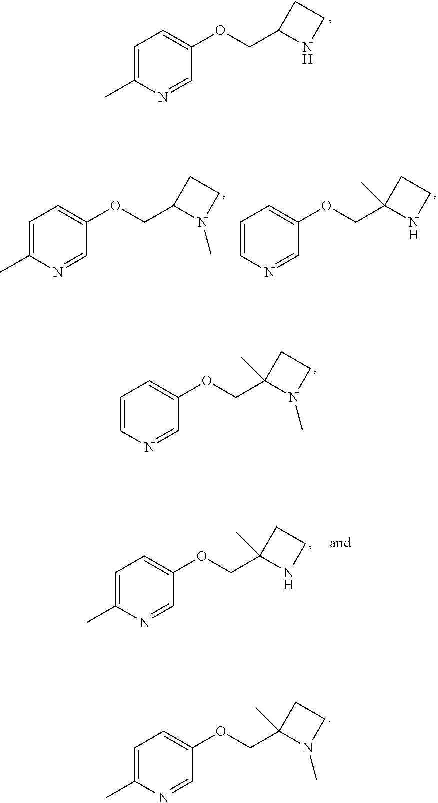

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the aerosol-modifying agent comprises unequal amounts of (R)- and (S)-enantiomer (i.e., is enriched in either the (R)- or (S)-enantiomer). In some embodiments, the aerosol-modifying agent predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the aerosol-modifying agent predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the aerosol-modifying agent comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

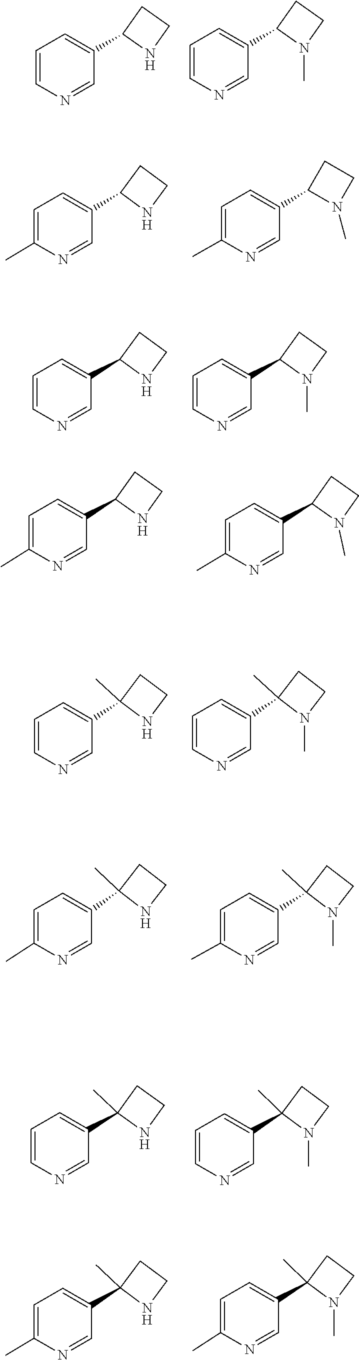

In some embodiments, the optionally substituted 3-(azetidin-2-ylmethoxy)pyridine is non-racemic, and has one of the following structures:

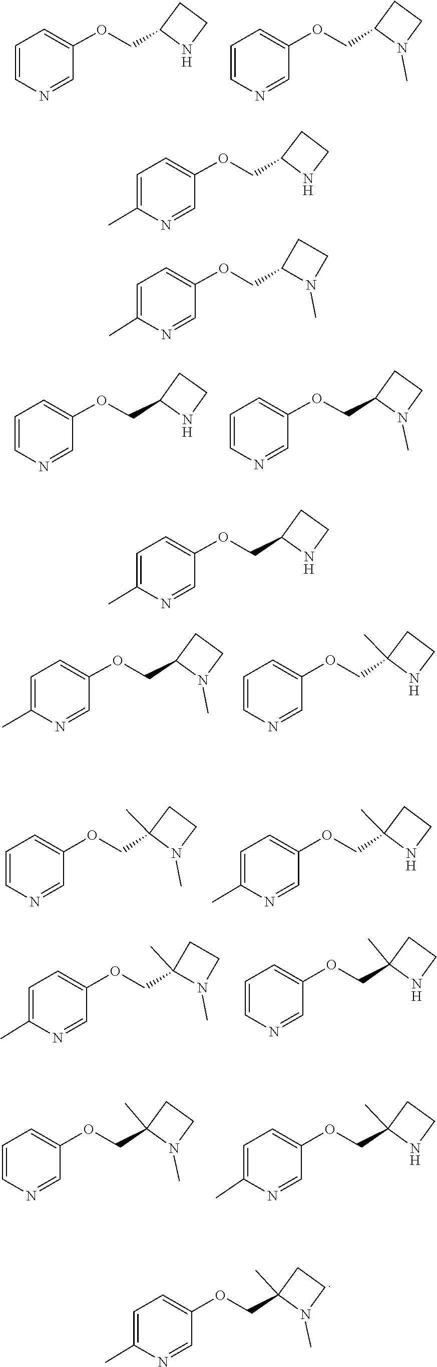

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the α₄β₂ subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the article (e.g. in the aerosol generating material) may vary. Typically, the optionally substituted 3-(azetidin-2-yl)pyridine or 3-(azetidin-2-ylmethoxy)pyridine, calculated as the free base, is present in a concentration of at least about 0.001% by weight of the article, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w, about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the article. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the article. One of skill in the art will recognize that the amount of any particular optionally substituted 3-(azetidin-2-yl)pyridine or 3-(azetidin-2-ylmethoxy)pyridine present in the article may vary based on the potency of the compound, the aerosol generating material matrix, and the desired physiological effect for the article.

In some embodiments, the amount of optionally substituted 3-(azetidin-2-yl)pyridine or 3-(azetidin-2-ylmethoxy)pyridine in the article is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the article is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

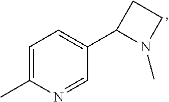

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

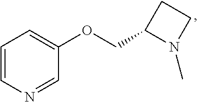

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the aerosol generating material as the free base, as a salt with a suitable acid, or in the form of an ion pair with an organic acid. Each of these forms is described further herein below.

Other Ingredients with Nicotinic Activity

In some embodiments, the substituted 3-(1-methylpyrrolidin-2-yl)pyridine, optionally substituted 3-(azetidin-2-yl)pyridine, or optionally 3-(azetidin-2-ylmethoxy)pyridine of the present disclosure is replaced with, or combined with, other active ingredients that provide the same general pharmacological profile and/or physiological effects of nicotine (i.e., an ingredient with nicotinic activity). Certain of these ingredients with nicotinic activity may be equipotent or even more potent than nicotine with respect to binding affinity to the nicotinic acetylcholine receptor and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example ingredients of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other ingredient with nicotinic activity is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

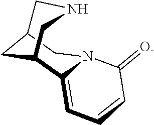

Cytisine is commercially available and has been utilized in post-Soviet states for more than 40 years as an aid to smoking cessation under the brand name Tabex (Sopharma AD). Cytisine is a partial agonist of the α₄β₂ nicotinic acetylcholine receptor.

In some embodiments, the other ingredient with nicotinic activity is varenicline or an analog thereof. Varenicline is commercially available as Chantix (Pfizer) and is a medication used as an aid for smoking cessation. Varenicline (CAS Registry No. 249296-44-4) has the structure:

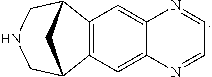 [SMILES: c1cnc2cc3c(cc2n1)[C@@H]1CNC[C@H]3C1]

Like cytisine, varenicline is a partial agonist of the α₄β₂ nicotinic acetylcholine receptor.

The quantity of the other ingredient with nicotinic activity present in the composition may vary. Typically, the other ingredient with nicotinic activity, calculated as the free base, is present in a concentration of at least about 0.001% by weight of the article, such as in a range from about 0.01% to about 10%. In some embodiments, the other ingredient with nicotinic activity is present in a concentration from about 0.05% w/w to about 5% by weight, such as, e.g., from about from about 0.05% w/w, about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the article. In some embodiments, the other ingredient with nicotinic activity is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the article. One of skill in the art will recognize that the amount of any particular other ingredient with nicotinic activity present in the aerosol generating material may vary based on the potency of the compound, the composition matrix, and the desired physiological effect for the composition.

The other active ingredient may be present in the article as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active ingredient exhibits sufficient stability, aqueous solubility, and oral bioavailability such that the free base is suitable for inclusion in the article. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine or other active ingredient is present substantially or completely as the free base. In such embodiments, one of skill in the art will recognize that the article is substantially free of acidic components. By “substantially free” it is meant that no acidic component (e.g., inorganic acid, organic acid, or acids capable of salt has been intentionally added, beyond trace amounts that may be present e.g., as an impurity in another component, or small amounts which may be present in certain flavor packages. For example, some embodiments can have less than 0.001% by weight of any acid component, or less than 0.0001%, or even 0% by weight of any acid component, based on the total weight of the article. In some embodiments, the article is completely free of any acid component (i.e., having 0% or having an amount below the limit of detection). In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally 3-(azetidin-2-ylmethoxy)pyridine, or other active ingredient can be provided using the types of ingredients and techniques set forth for nicotine in U.S. Pat. No. 2,033,909 to Cox et al. and Perfetti, Beitrage Tabakforschung Int., 12:43-54 (1983), which are incorporated herein by reference Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active ingredient salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

### Organic Acid

In some embodiments, the article (e.g., within the aerosol-modifying agent release component and/or aerosol generating material) comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid. In some embodiments, the organic acid is benzoic acid.

Further non-limiting examples of organic acids which may be useful in certain embodiments include 2-(4-isobutylphenyl) propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid
monooctyl succinate
tocopherol succinate
monomenthyl succinate
monomenthyl glutarate
norbixin ((2E,4E,6E,8E,10E,12E,14E,16E,18E)-4,8,13,17
tetramethylicosa-2,4,6,8,10,12,14,16,18-nonaenedioic acid)
bixin ((2E,4E,6E,8E,10E,12E,14E,16Z,18E)-20-methoxy
4,8,13,17-tetramethyl-20-oxoicosa-2,4,6,8,10,12,14,16,18-
nonaenoic acid)

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art. In some embodiments, the acid is benzoic acid. In some embodiments, the acid is levulinic acid. In some embodiments, the acid is lactic acid. In some embodiments, the acid is one or more of benzoic acid, lactic acid, and levulinic acid.

In some embodiments, more than one organic acid may be present. For example, the aerosol-modifying agent may comprise two, or three, or four, or more organic acids. Accordingly, reference herein to “an organic acid” contemplates mixtures of two or more organic acids. The relative amounts of the multiple organic acids may vary. For example, an aerosol-modifying agent may comprise equal amounts of two, or three, or more organic acids, or may comprise different relative amounts. Without wishing to be bound by theory, it is believed that a combination of different organic acids may provide a concentration of any single organic acid in the composition which remains below the threshold which would be found objectionable from a sensory perspective.

The amount of organic acid present in the article (e.g., in the aerosol-modifying agent release component and/or the aerosol generating material), relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), may vary. In some embodiments, the article comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine).

In some embodiments, the article (e.g., in the aerosol-modifying agent release component and/or the aerosol generating material) comprises from about 2 to about 10, or from about 2 to about 5 molar equivalents of the organic acid relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), on a free-base basis. In some embodiments, the organic acid is present in a molar ratio with the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine) from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the article (e.g., in the aerosol-modifying agent release component and/or the aerosol generating material) comprises one or more of the organic acids described herein above. The substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine) and the one or more organic acids may be present independently, in the form of a salt, or as a cocrystal. In some embodiments, the aerosol-modifying agent comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine, 3-(azetidin-2-yl)pyridine, or 3-(azetidin-2-ylmethoxy)pyridine.

In some embodiments, the article (e.g., in the aerosol-modifying agent release component and/or the aerosol generating material) comprises a lactic acid salt of the substituted 3-(1-methylpyrrolidin-2-yl)pyridine, 3-(azetidin-2-yl)pyridine, or 3-(azetidin-2-ylmethoxy)pyridine.

In some embodiments, the article (e.g., in the aerosol-modifying agent release component and/or the aerosol generating material) comprises a levulinic acid salt of the substituted 3-(1-methylpyrrolidin-2-yl)pyridine, 3-(azetidin-2-yl)pyridine, or 3-(azetidin-2-ylmethoxy)pyridine.

In some embodiments, the article (e.g., in the aerosol-modifying agent release component and/or the aerosol generating material) comprises a galactaric acid salt of the substituted 3-(1-methylpyrrolidin-2-yl)pyridine, 3-(azetidin-2-yl)pyridine, or 3-(azetidin-2-ylmethoxy)pyridine.

In some embodiments, the article (e.g., in the aerosol-modifying agent release component and/or the aerosol generating material) comprises a tartaric acid salt of the substituted 3-(1-methylpyrrolidin-2-yl)pyridine, 3-(azetidin-2-yl)pyridine, or 3-(azetidin-2-ylmethoxy)pyridine.

The stoichiometry of such lactate, levulinate, tartrate, and galactarate salts may vary. Accordingly, the molar ratio of lactic, levulinic, tartaric, or galactaric acid to substituted 3-(1-methylpyrrolidin-2-yl)pyridine, 3-(azetidin-2-yl)pyridine, or 3-(azetidin-2-ylmethoxy)pyridine may be in a range from about 3:1 to about 1:3, including any whole or fractional value in between, such as for example about 2:1, about 1.5:1, about 1:1, about 1:1.5, or about 1:2.

In some embodiments, the article may be substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection).

### Resin Complex

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally 3-(azetidin-2-ylmethoxy)pyridine is present in the form of a polymer complex, the composition further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally 3-(azetidin-2-ylmethoxy)pyridine, or other active ingredient in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (β-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally 3-(azetidin-2-ylmethoxy)pyridine molecule, or other active ingredient within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active ingredient, and wherein a second coformer does not transfer a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active ingredient. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally 3-(azetidin-2-ylmethoxy)pyridine, or other active ingredient and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other nicotinic active ingredient is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the composition as disclosed herein, and/or upon oral use of said composition (e.g., upon contact with saliva), the substituted 3-(1-methylpyrrolidin-2-yl)pyridine, optionally substituted 3-(azetidin-2-yl)pyridine, optionally 3-(azetidin-2-ylmethoxy)pyridine, or other active ingredient and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed article, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally 3-(azetidin-2-ylmethoxy)pyridine, or other active ingredient, the concentration of the acid or conjugate base of the acid present in the article, the moisture content of the aerosol generating material, the ionic strength of the aerosol generating material, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active ingredient through the oral mucosa. However, as described above, in some embodiments, the properties of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active ingredient are such that no ion pairing is required, and accordingly, the composition is substantially or completely free of any ion pairing. By “substantially free” it is meant that no measurable degree of ion pairing is present.

In embodiments where ion pairing is desired, the article (e.g., in the aerosol-modifying agent release component and/or the aerosol generating material) comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the composition of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the article (e.g., in the aerosol-modifying agent release component and/or the aerosol generating material) may be characterized as substantially or completely free of organic acids (i.e., having less than 0.001% by weight of organic acid, or less than 0.0001%, or even 0% by weight of organic acid, based on the total weight of the article, or as having an amount of organic acid below the limit of detection). This is not to be interpreted as meaning that the article is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the article, relative to the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active ingredient, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally 3-(azetidin-2-ylmethoxy)pyridine, or other active ingredient that is ion paired with the organic acid increases. This typically increases the partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient in the form of an ion pair, into octanol versus water as measured by the log P (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active ingredient, calculated as the free base of the substituted 3-(1-methylpyrrolidin-2-yl)pyridine, optionally substituted 3-(azetidin-2-yl)pyridine, optionally 3-(azetidin-2-ylmethoxy)pyridine, or other active ingredient.

In some embodiments, the article comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active ingredient on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally 3-(azetidin-2-ylmethoxy)pyridine, or other active ingredient from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the article comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally 3-(azetidin-2-ylmethoxy)pyridine, or other active ingredient. In some embodiments, the molar ratio of the total amount of benzoate to the substituted 3-(1-methylpyrrolidin-2-yl)pyridine, optionally substituted 3-(azetidin-2-yl)pyridine, optionally 3-(azetidin-2-ylmethoxy)pyridine, or other active ingredient is about 3.2 or about 4.8.

In some embodiments, the organic acid inclusion is sufficient to provide a pH of from about 4.0 to about 9.0, such as from about 4.5 to about 7.0, or from about 5.5 to about 7.0, from about 4.0 to about 5.5, or from about 7.0 to about 9.0. Reference herein to “a pH” means the pH of an aqueous solution of the aerosol generating material or aerosol-modifying agent release component prepared by dissolving or suspending 5 grams of aerosol generating material or aerosol-modifying agent release component in 95 grams of water and measuring the pH of the resulting solution with a calibrated pH meter.

In some embodiments, the organic acid inclusion is sufficient to provide a pH of from about 4.5 to about 6.5, for example, from about 4.5, about 5.0, or about 5.5, to about 6.0, or about 6.5. In some embodiments, the desired pH is from about 4.5 to about 6.5, and the organic acid is provided in a quantity sufficient to provide such a pH. In some embodiments, the organic acid is provided in a quantity sufficient to provide a pH of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the aerosol-modifying agent release component to the desired value. In some embodiments, a buffer (e.g., a buffer as described herein below) is added to the aerosol-modifying agent release component to the desired value, and/or to maintain the pH of the aerosol-modifying agent release component at the desired value.

In some embodiments, the aerosol-modifying agent release component and/or the aerosol generating material further comprises a solubility enhancer to increase the solubility of one or more of the organic acid or salt thereof. Suitable solubility enhancers include, but are not limited to, humectants as described herein, such as glycerol or propylene glycol.

In some embodiments, the article (e.g., in the aerosol-modifying agent release component and/or the aerosol generating material) further comprises one or more flavorants, and/or one or more volatile substances which modify at least one property of the aerosol. In some embodiments, the article (e.g., in the aerosol-modifying agent and/or the aerosol generating material) further comprises an additional active ingredient as described herein above.

### Form of Aerosol-Modifying Agent Release Component

The form in which the aerosol-modifying agent release component is present in the aerosol generating article may vary. In some embodiments, the aerosol-modifying agent release component is present in the form of one or more capsules comprising a shell or barrier material encapsulating the aerosol-modifying agent. That is, the encapsulating or barrier material creates a shell around a core that comprises the aerosol-modifying agent. The shell or barrier material generally comprises a gelling agent, a bulking agent, a filler, a buffer, a coloring agent, a plasticizer, or a combination thereof.

Examples of suitable gelling agents include a polysaccharide or cellulosic gelling agent, a gelatin, a gum, a gel, a wax or a mixture thereof. Suitable polysaccharides include alginates, dextrans, maltodextrins, cyclodextrins and pectins. Suitable alginates include, for instance, a salt of alginic acid, an esterified alginate or glyceryl alginate. Salts of alginic acid include ammonium alginate, triethanolamine alginate, and group I or II metal ion alginates like sodium, potassium, calcium and magnesium alginate. Esterified alginates include propylene glycol alginate and glyceryl alginate. In an embodiment, the barrier material is sodium alginate and/or calcium alginate. Suitable cellulosic materials include methyl cellulose, ethyl cellulose, hydroxyethyl cellulose, hydroxypropyl cellulose, carboxymethyl cellulose, cellulose acetate and cellulose ethers. The gelling agent may comprise one or more modified starches. The gelling agent may comprise carrageenans. Suitable gums include agar, gellan gum, gum Arabic, pullulan gum, mannan gum, gum ghatti, gum tragacanth, Karaya, locust bean, acacia gum, guar, quince seed and xanthan gums. Suitable gels include agar, agarose, carrageenans, furoidan and furcellaran. Suitable waxes include carnauba wax. In some cases, the gelling agent may comprise carrageenans and/or gellan gum; these gelling agents are particularly suitable for inclusion as the gelling agent as the pressure required to break the resulting capsules is particularly suitable.

The barrier material may comprise one or more bulking agents, such as starches, modified starches (such as oxidized starches) and sugar alcohols such as maltitol.

The barrier material may comprise a coloring agent which renders easier the location of the capsule within the aerosol generating device during the manufacturing process of the aerosol generating device. The coloring agent is preferably chosen among colorants and pigments.

The barrier material may further comprise at least one buffer, such as a citrate or phosphate compound.

The barrier material may further comprise at least one plasticizer, which may be glycerol, sorbitol, maltitol, triacetin, polyethylene glycol, propylene glycol or another polyalcohol with plasticizing properties, and optionally one acid of the monoacid, diacid or triacid type, especially citric acid, fumaric acid, malic acid, and the like. The amount of plasticizer ranges from 1% to 30% by weight, preferably from 2% to 15% by weight, and even more preferably from 3 to 10% by weight of the total dry weight of the shell.

The barrier material may also comprise one or more filler materials. Suitable filler materials include comprising starch derivatives such as dextrin, maltodextrin, cyclodextrin (alpha, beta or gamma), or cellulose derivatives such as hydroxypropylmethylcellulose (HPMC), hydroxypropylcellulose (HPC), methylcellulose (MC), carboxymethylcellulose (CMC), polyvinyl alcohol, polyols or mixture thereof. Dextrin is a preferred filler. The amount of filler in the shell is at most 98.5%, preferably from 25 to 95% more preferably from 40 to 80% and even more preferably from 50 to 60% by weight on the total dry weight of the shell.

The capsule shell may additionally comprise a hydrophobic outer layer which reduces the susceptibility of the capsule to moisture-induced degradation. The hydrophobic outer layer is suitably selected from the group comprising waxes, especially carnauba wax, candelilla wax or beeswax, carbowax, shellac (in alcoholic or aqueous solution), ethyl cellulose, hydroxypropyl methyl cellulose, hydroxypropylcellulose, latex composition, polyvinyl alcohol, or a combination thereof. In some embodiments, the at least one moisture barrier agent is ethyl cellulose or a mixture of ethyl cellulose and shellac.

The core may also comprise a solvent which dissolves the aerosol-modifying agent. Any suitable solvent may be used. Suitable solvents include, but are not limited to, short or medium chain fats and oils, glycerin, propylene glycol, tri-esters of glycerol such as C2-C12 triglycerides. For example, the solvent may comprise medium chain triglycerides (MCT—C8-C12), which may be derived from palm oil and/or coconut oil.

In some embodiments, the barrier material is frangible. The capsule is crushed or otherwise fractured or broken by the user to release the encapsulated aerosol modifier. Typically, the capsule is broken immediately prior to heating being initiated but the user can select when to release the aerosol modifier. The term “breakable capsule” refers to a capsule, wherein the shell can be broken by means of a pressure to release the core; more specifically the shell can be ruptured such as under the pressure imposed by the user's fingers when the user wants to release the core of the capsule, or can be ruptured upon insertion of a consumable into a device, such as through mechanical pressure applied by interaction between the consumable and sidewalls of the receiving chamber of the device or piercing the capsule with e.g., a pin heater. In some cases, the barrier material is heat resistant. That is to say, in some cases, the barrier will not rupture, melt or otherwise fail at the temperature reached at the capsule site during operation of the aerosol provision device. Illustratively, a capsule located in a mouthpiece may be exposed to temperatures in the range of 30° C. to 100° C. for example, and the barrier material may continue to retain the liquid core up to at least about 50 C to 120° C.

In some embodiments, the capsule releases the core composition on heating, for example by melting of the barrier material or by capsule swelling leading to rupture of the barrier material.

The capsules may present a crush strength from about 4.5 N to about 40 N, more preferably from about 5 N to about 30 N or to about 28 N (for instance about 9.8 N to about 24.5 N). The capsule burst strength can be measured using a force gauge to measure the force at which the capsule bursts when pressed between two flat metal plates. A suitable measurement device is the Sauter FK 50 force gauge with a flat headed attachment, which can be used to crush the capsule against a flat, hard surface having a surface similar to the attachment.

In some embodiments, the one or more capsules are spherical and have a diameter in a range from about 0.4 mm to about 10 mm. In some embodiments, the capsules have a diameter of at least about 0.4 mm, 0.6 mm, 0.8 mm, 1.0 mm, 2.0 mm, 2.5 mm, 2.8 mm or 3.0 mm. The diameter of the capsules may be less than about 10.0 mm, 8.0 mm, 7.0 mm, 6.0 mm, 5.5 mm, 5.0 mm, 4.5 mm, 4.0 mm, 3.5 mm or 3.2 mm. Illustratively, the capsule diameter maybe in the range of about 0.4 mm to about 10.0 mm, about 0.8 mm to about 6.0 mm, about 2.5 mm to about 5.5 mm or about 2.8 mm to about 3.2 mm. In some embodiments, the capsule may have a diameter of about 3.0 mm. These sizes are particularly suitable for incorporation of the capsule into an article as described herein below. In other embodiments, other shapes and sizes of capsule can be used.

The total weight of a capsule may be in the range of about 1 mg to about 100 mg, suitably about 5 mg to about 60 mg, about 8 mg to about 50 mg, about 10 mg to about 20 mg, or about 12 mg to about 18 mg.

The total weight of the core formulation may be in the range of about 2 mg to about 90 mg, suitably about 3 mg to about 70 mg, about 5 mg to about 25 mg, about 8 mg to about 20 mg, or about 10 mg to about 15 mg.

In some embodiments, the capsules may include from about 0.01 to about 5 mg of the aerosol-modifying agent.

The location of such capsules within the aerosol generating article may vary and is described further herein below with respect to aerosol generating articles.

In some embodiments, in addition to or as an alternative to capsules, the aerosol-modifying agent may be absorbed or adsorbed in or on a wrapping material to form the aerosol-modifying agent release component. In some embodiments, in addition to or as an alternative to capsules, the aerosol-modifying agent may be present in an aerosol generating article in powder, thread, or granule form.

### Aerosol Generating Article

In another aspect is provided an aerosol generating article comprising an aerosol generating material and an aerosol-modifying agent, each as described herein. An “article” in this context is a component that includes or contains in use the aerosol generating material, which is heated to volatilize the aerosol generating material, and optionally includes other components. A user may insert the article into an aerosol generating device before it is heated to produce an aerosol, which the user subsequently inhales. The article may be, for example, of a predetermined or specific size that is configured to be placed within a heating chamber of a device which is sized to receive the article. Such aerosol generating articles may further comprise additional elements, such as a wrapping material, a filter, a cooling element, a mouthpiece, or combinations thereof.

In some embodiments, the aerosol generating article comprises a wrapper which at least partially surrounds the article, and the aerosol-modifying agent or a portion thereof is absorbed or adsorbed in or on the wrapping material.

In some embodiments, the aerosol generating article comprises, in addition to the aerosol generating material, a filter, a cooling element, and a mouthpiece, and the wrapper at least partially surrounds one or more of these additional components of the article. In some embodiments, the wrapper may surround the perimeter of each of these components. In some embodiments, the wrapper may form a tube disposed around the rod of aerosol generating material.

The wrapper may have a thickness of between about 10 μm and 50 μm, suitably between about 15 μm and 45 μm or between about 20 μm and 40 μm. In some embodiments, the wrapper may comprise a paper layer, and in some cases this may have a basis weight of at least about 10 g·m⁻², 15 g·m⁻², 20 g·m⁻² or 25 g·m⁻² to about 50 g·m⁻², 45 g·m⁻², 40 g·m⁻² or 35 g·m⁻². In some embodiments, the wrapper may comprise a non-combustible layer, such as a metallic foil. Suitably, the wrapper may comprise an aluminum foil layer, which may have a thickness between about 3 μm and 15 μm, suitably between about 5 μm and 10 μm, suitably about 6 μm. The wrapper may comprise a laminate structure, and in some embodiments, the laminate structure may comprise a least one paper layer and at least one non-combustible layer.

In some embodiments, ventilation apertures are provided in the wrapper. In some embodiments, the ventilation ratio provided by the holes (i.e., the amount of inhaled air flowing through the ventilation holes as a percentage of the aerosol volume) may be between about 5% and 85%, suitably at least 20%, 35%, 50% or 60%. The ventilation apertures may be provided in the wrapper in the portion that surrounds one or more of a filter, a cooling element, and a mouthpiece.

In some embodiments, the aerosol generating article is substantially cylindrical and has a total length of between about 71 mm and 95 mm. In some embodiments, the aerosol generating material is in the form of a cylindrical rod having a diameter of between about 5.0 mm and 6.0 mm.

In some embodiments, the aerosol generating article further comprises a mouthpiece having an upstream end and a downstream end, the mouthpiece comprising a hollow tubular element formed from filamentary tow at the downstream end of the mouthpiece. In some embodiments, the mouthpiece comprises a body of material in the form of a cylinder having a longitudinal axis, and the aerosol-modifying agent release component is present in the form of one or more capsules as described herein, and wherein the one or more capsules are embedded within the body of material such that the one or more capsules are surrounded on all sides by the material forming the body of material.

### Aerosol Generating Device

In another aspect is provided an aerosol generating device. As used herein, an “aerosol generating device” is a device configured to receive an aerosol generating article as disclosed herein and to heat the article to provide an aerosol. Such devices may also be referred to as a non-combustible aerosol provision device or system. The combination of the aerosol generating device and aerosol generating article is referred to herein as an aerosol generating assembly.

According to the present disclosure, a “non-combustible” aerosol provision system is one where a constituent aerosol-generating material of the aerosol provision system (or component thereof) is not combusted or burned in order to facilitate delivery of at least one substance to a user. In some examples, the delivery system is a non-combustible aerosol provision system, such as a powered non-combustible aerosol provision system.

In some examples, the non-combustible aerosol provision system, such as a non-combustible aerosol provision device thereof, may comprise a power source and a controller. The power source may, for example, be an electric power source or an exothermic power source. In some examples, the exothermic power source comprises a carbon substrate which may be energized so as to distribute power in the form of heat to an aerosol-generating material or to a heat transfer material in proximity to the exothermic power source.

In some examples, the non-combustible aerosol provision system may comprise an area for receiving the consumable, an aerosol generator, an aerosol generation area, a housing, a mouthpiece, a filter and/or an aerosol-modifying agent.

In some examples, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

An aerosol generator is an apparatus configured to cause aerosol to be generated from the aerosol-generating material. In some examples, the aerosol generator is a heater configured to subject the aerosol-generating material to heat energy, so as to release one or more volatiles from the aerosol-generating material to form an aerosol. In some examples, the aerosol generator is configured to cause an aerosol to be generated from the aerosol-generating material without heating. For example, the aerosol generator may be configured to subject the aerosol-generating material to one or more of vibration, increased pressure, or electrostatic energy.

Example aerosol generator technologies include an induction heater, a laser heater, a plasma heater, a microfluidic heater, a convection heater, allotrope of carbon heater, carbon foam heater, a radio-frequency heater, an electro-resistive heater, and a halogen heater.

In some embodiments, the coil is configured to, in use, cause heating of at least one electrically conductive heating element, so that heat energy is conductible from the at least one electrically conductive heating element to the aerosol generating material to thereby cause heating of the aerosol generating material. In some examples, the coil is configured to generate, in use, a varying magnetic field for penetrating at least one heating element, to thereby cause induction heating and/or magnetic hysteresis heating of the at least one heating element. In such an arrangement, the heating element may be termed a “susceptor” as defined herein. A coil that is configured to generate, in use, a varying magnetic field for penetrating at least one electrically conductive heating element, to thereby cause induction heating of the at least one electrically conductive heating element, may be termed an “induction coil” or “inductor coil”.

The device may include the heating element(s), for example electrically conductive heating element(s), and the heating element(s) may be suitably located or locatable relative to the coil to enable such heating of the heating element(s). The heating element(s) may be in a fixed position relative to the coil. Alternatively, the at least one heating element, for example at least one electrically conductive heating element, may be included in the article for insertion into a heating zone of the device, wherein the article also comprises the aerosol generating material and is removable from the heating zone after use. Alternatively, both the device and such an article may comprise at least one respective heating element, for example at least one electrically conductive heating element, and the coil may be to cause heating of the heating element(s) of each of the device and the article when the article is in the heating zone.

In some embodiments, the coil is helical. In some examples, the coil encircles at least a part of a heating zone of the device that is configured to receive the aerosol generating material. In some embodiments, the coil is a helical coil that encircles at least a part of the heating zone.

In some embodiments, the device comprises an electrically conductive heating element that at least partially surrounds the heating zone, and the coil is a helical coil that encircles at least a part of the electrically conductive heating element. In some embodiments, the electrically conductive heating element is tubular. In some examples, the coil is an inductor coil.

The aerosol generating device may further comprise a controller which drives the induction heater, wherein the controller is programmed with selectable heating profiles, and wherein the device comprises a user interface, allowing the user to select the desired heating profile in use. That is, the controller may be programmed with a least two pre-determined heat profiles, and the user can select which of these is desired in use. The heat profiles may differ from each other in a number of ways, including but not limited to the rate of heating, the period of heating, and the maximum temperature. Where there are two or more heating zones, the heating profiles may differ in the behavior of only one zone, or in the behavior of each zone.

As noted above, in some cases, the susceptor defines a cylindrical chamber into which the article is inserted in use, so that the aerosol generating material is heated by the susceptor. The cylindrical chamber length may be from about 40 mm to 60 mm, about 40 mm to 50 mm or about 40 mm to 45 mm, or about 44.5 mm. The cylindrical chamber diameter may be from about 5.0 mm to 6.5 mm, suitably about 5.35 mm to 6.0 mm, suitably about 5.5 mm to 5.6 mm, suitably about 5.55 mm. In some embodiments, the aerosol generating material may fill at least about 85% of a void defined by the susceptor.

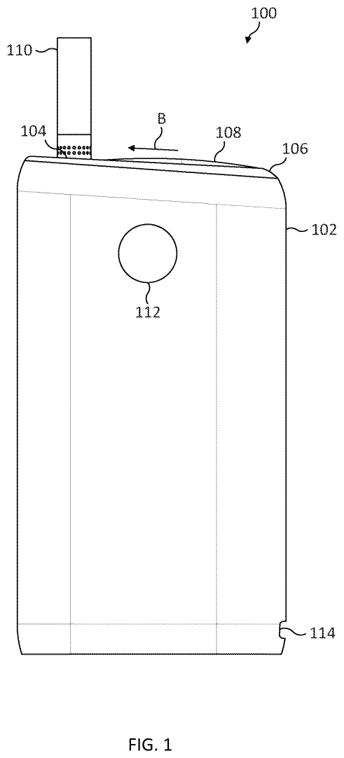

Referring now to the Figures, there is illustrated in FIG. 1 an embodiment of an aerosol generating device 100 for generating aerosol from an aerosol generating material as disclosed herein. In broad outline, the device 100 may be used to heat a replaceable article 110 comprising the aerosol generating material and the aerosol-modifying agent, to generate an aerosol or other inhalable medium which is inhaled by a user of the device 100.

The device 100 comprises a housing 102 (in the form of an outer cover) which surrounds and houses various components of the device 100. The device 100 has an opening 104 in one end, through which the article 110 may be inserted for heating by a heating assembly. In use, the article 110 may be fully or partially inserted into the heating assembly where it may be heated by one or more components of the heater assembly.

The device 100 of this embodiment comprises a first end member 106 which comprises a lid 108 which is moveable relative to the first end member 106 to close the opening 104 when no article 110 is in place. In FIG. 1, the lid 108 is shown in an open configuration, however the cap 108 may move into a closed configuration. For example, a user may cause the lid 108 to slide in the direction of arrow “B”.

The device 100 may also include a user-operable control element 112, such as a button or switch, which operates the device 100 when pressed. For example, a user may turn on the device 100 by operating the switch 112. In some cases, different heat profiles may be accessed through predetermined interactions with the switch (e.g., number of presses of switch, or length of press).

The device 100 may also comprise an electrical component, such as a socket/port 114, which can receive a cable to charge a battery of the device 100. For example, the socket 114 may be a charging port, such as a USB charging port. In some embodiments, the socket 114 may be used additionally or alternatively to transfer data between the device 100 and another device, such as a computing device.

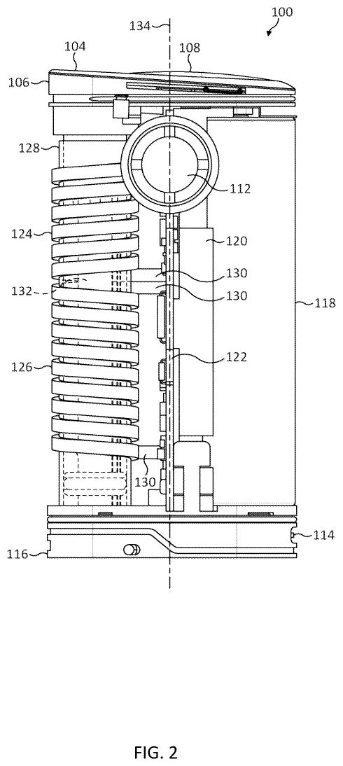

FIG. 2 depicts the device 100 of FIG. 1 with the outer cover 102 removed and without an article 110 present. The device 100 defines a longitudinal axis 134.

As shown in FIG. 2, the first end member 106 is arranged at one end of the device 100 and a second end member 116 is arranged at an opposite end of the device 100. The first and second end members 106, 116 together at least partially define end surfaces of the device 100. For example, the bottom surface of the second end member 116 at least partially defines a bottom surface of the device 100. The end member 116 may further house one or more electrical components, such as a socket/port 114. The socket 114 in this example is a female USB charging port.

Edges of the outer cover 102 may also define a portion of the end surfaces. In this example, the lid 108 also defines a portion of a top surface of the device 100.

The end of the device closest to the opening 104 may be known as the proximal end (or mouth end) of the device 100 because, in use, it is closest to the mouth of the user. In use, a user inserts an article 110 into the opening 104, operates the user control 112 to begin heating the aerosol generating material and draws on the aerosol generated in the device. This causes the aerosol to flow through the device 100 along a flow path towards the proximal end of the device 100.

The other end of the device furthest away from the opening 104 may be known as the distal end of the device 100 because, in use, it is the end furthest away from the mouth of the user. As a user draws on the aerosol generated in the device, the aerosol flows away from the distal end of the device 100.

The device 100 further comprises a power source 118. The power source 118 may be, for example, a battery, such as a rechargeable battery or a non-rechargeable battery. Examples of suitable batteries include, for example, a lithium battery (such as a lithium-ion battery), a nickel battery (such as a nickel-cadmium battery), and an alkaline battery. The battery is electrically coupled to the heating assembly to supply electrical power when required and under control of a controller (not shown) to heat the aerosol generating material. In this example, the battery is connected to a central support 120 which holds the battery 118 in place.

The device further comprises at least one electronics module 122. The electronics module 122 may comprise, for example, a printed circuit board (PCB). The PCB 122 may support at least one controller, such as a processor, and memory. The PCB 122 may also comprise one or more electrical tracks to electrically connect together various electronic components of the device 100. For example, the battery terminals may be electrically connected to the PCB 122 so that power can be distributed throughout the device 100. The socket 114 may also be electrically coupled to the battery via the electrical tracks.

In the example device 100, the heating assembly is an inductive heating assembly and comprises various components to heat the aerosol generating material of the article 110 via an inductive heating process. Induction heating is a process of heating an electrically conducting object (such as a susceptor) by electromagnetic induction. An induction heating assembly may comprise an inductive element, for example, one or more inductor coils, and a device for passing a varying electric current, such as an alternating electric current, through the inductive element. The varying electric current in the inductive element produces a varying magnetic field. The varying magnetic field penetrates a susceptor suitably positioned with respect to the inductive element and generates eddy currents inside the susceptor. The susceptor has electrical resistance to the eddy currents, and hence the flow of the eddy currents against this resistance causes the susceptor to be heated by Joule heating. In cases where the susceptor comprises ferromagnetic material such as iron, nickel or cobalt, heat may also be generated by magnetic hysteresis losses in the susceptor, i.e., by the varying orientation of magnetic dipoles in the magnetic material as a result of their alignment with the varying magnetic field. In inductive heating, as compared to heating by conduction for example, heat is generated inside the susceptor, allowing for rapid heating. Further, there need not be any physical contact between the inductive heater and the susceptor, allowing for enhanced freedom in construction and application.

The induction heating assembly of the example device 100 comprises a susceptor arrangement 132 (herein referred to as “a susceptor”), a first inductor coil 124 and a second inductor coil 126. The first and second inductor coils 124, 126 are made from an electrically conducting material. In this example, the first and second inductor coils 124, 126 are made from Litz wire/cable which is wound in a helical fashion to provide helical inductor coils 124, 126. Litz wire comprises a plurality of individual wires which are individually insulated and are twisted together to form a single wire. Litz wires are designed to reduce the skin effect losses in a conductor. In the example device 100, the first and second inductor coils 124, 126 are made from copper Litz wire which has a rectangular cross section. In other examples the Litz wire can have other shape cross sections, such as circular.

The first inductor coil 124 is configured to generate a first varying magnetic field for heating a first section of the susceptor 132 and the second inductor coil 126 is configured to generate a second varying magnetic field for heating a second section of the susceptor 132. In this example, the first inductor coil 124 is adjacent to the second inductor coil 126 in a direction along the longitudinal axis 134 of the device 100 (that is, the first and second inductor coils 124, 126 to not overlap). The susceptor arrangement 132 may comprise a single susceptor, or two or more separate susceptors. Ends 130 of the first and second inductor coils 124, 126 can be connected to the PCB 122.

It will be appreciated that the first and second inductor coils 124, 126, in some examples, may have at least one characteristic different from each other. For example, the first inductor coil 124 may have at least one characteristic different from the second inductor coil 126. More specifically, in one example, the first inductor coil 124 may have a different value of inductance than the second inductor coil 126. In FIG. 2, the first and second inductor coils 124, 126 are of different lengths such that the first inductor coil 124 is wound over a smaller section of the susceptor 132 than the second inductor coil 126. Thus, the first inductor coil 124 may comprise a different number of turns than the second inductor coil 126 (assuming that the spacing between individual turns is substantially the same). In yet another example, the first inductor coil 124 may be made from a different material to the second inductor coil 126. In some examples, the first and second inductor coils 124, 126 may be substantially identical.

In this example, the first inductor coil 124 and the second inductor coil 126 are wound in opposite directions. This can be useful when the inductor coils are active at different times. For example, initially, the first inductor coil 124 may be operating to heat a first section of the article 110, and at a later time, the second inductor coil 126 may be operating to heat a second section of the article 110. Winding the coils in opposite directions helps reduce the current induced in the inactive coil when used in conjunction with a particular type of control circuit. In FIG. 2, the first inductor coil 124 is a right-hand helix and the second inductor coil 126 is a left-hand helix. However, in another embodiment, the inductor coils 124, 126 may be wound in the same direction, or the first inductor coil 124 may be a left-hand helix and the second inductor coil 126 may be a right-hand helix.

The susceptor 132 of this example is hollow and therefore defines a receptacle within which aerosol generating material is received. For example, the article 110 can be inserted into the susceptor 132. In this example the susceptor 120 is tubular, with a circular cross section.

The device 100 of FIG. 2 further comprises an insulating member 128 which may be generally tubular and at least partially surround the susceptor 132. The insulating member 128 may be constructed from any insulating material, such as plastic for example. In this particular embodiment, the insulating member is constructed from polyether ether ketone (PEEK). The insulating member 128 may help insulate the various components of the device 100 from the heat generated in the susceptor 132.

The insulating member 128 can also fully or partially support the first and second inductor coils 124, 126. For example, as shown in FIG. 2, the first and second inductor coils 124, 126 are positioned around the insulating member 128 and are in contact with a radially outward surface of the insulating member 128. In some examples the insulating member 128 does not abut the first and second inductor coils 124, 126. For example, a small gap may be present between the outer surface of the insulating member 128 and the inner surface of the first and second inductor coils 124, 126.

In a specific embodiment, the susceptor 132, the insulating member 128, and the first and second inductor coils 124, 126 are coaxial around a central longitudinal axis of the susceptor 132.

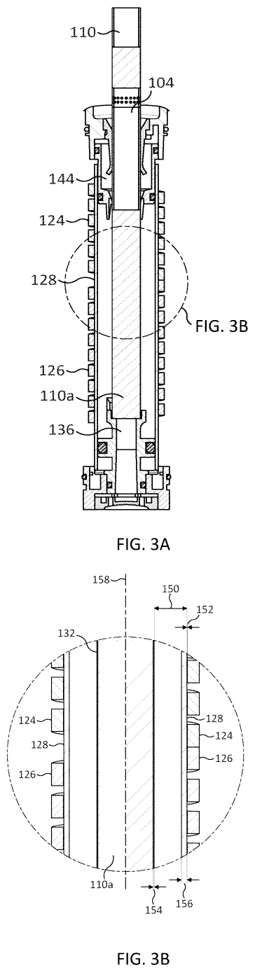

FIG. 3A depicts a cross section of a portion of the device 100 of FIG. 1. FIG. 3B depicts a close-up of a region of FIG. 3A. FIGS. 3A and 3B show the article 110 received within the susceptor 132, where the article 110 is dimensioned so that the outer surface of the article 110 abuts the inner surface of the susceptor 132. This ensures that the heating is most efficient. The article 110 of this example comprises an aerosol generating material 110a as disclosed herein. The aerosol generating material 110 a is positioned within the susceptor 132. The article 110 may also comprise other components such as a filter, wrapping materials and/or a cooling structure.

As shown in FIG. 3A, the device 100 can include an expansion chamber 144 which extends away from a proximal end of the susceptor 132 towards the opening 104 of the device. The expansion chamber 144 is connected to the end member 106. The device 100 further comprises a support 136 which engages one end of the susceptor 132 to hold the susceptor 132 in place. The support 136 is connected to the second end member 116.

FIG. 3B shows that the outer surface of the susceptor 132 is spaced apart from the inner surface of the inductor coils 124, 126 by a distance 150, measured in a direction perpendicular to a longitudinal axis 158 of the susceptor 132. In one particular embodiment, the distance 150 is about 3 mm to 4 mm, about 3-3.5 mm, or about 3.25 mm.

FIG. 3B further shows that the outer surface of the insulating member 128 is spaced apart from the inner surface of the inductor coils 124, 126 by a distance 152, measured in a direction perpendicular to a longitudinal axis 158 of the susceptor 132. In one particular embodiment, the distance 152 is about 0.05 mm. In another embodiment, the distance 152 is substantially 0 mm, such that the inductor coils 124, 126 abut and touch the insulating member 128.

In one embodiment, the susceptor 132 has a wall thickness 154 of about 0.025 mm to 1 mm, or about 0.05 mm.

In one embodiment, the susceptor 132 has a length of about 40 mm to 60 mm, about 40-45 mm, or about 44.5 mm.

In one embodiment, the insulating member 128 has a wall thickness 156 of about 0.25 mm to 2 mm, 0.25 to 1 mm, or about 0.5 mm.

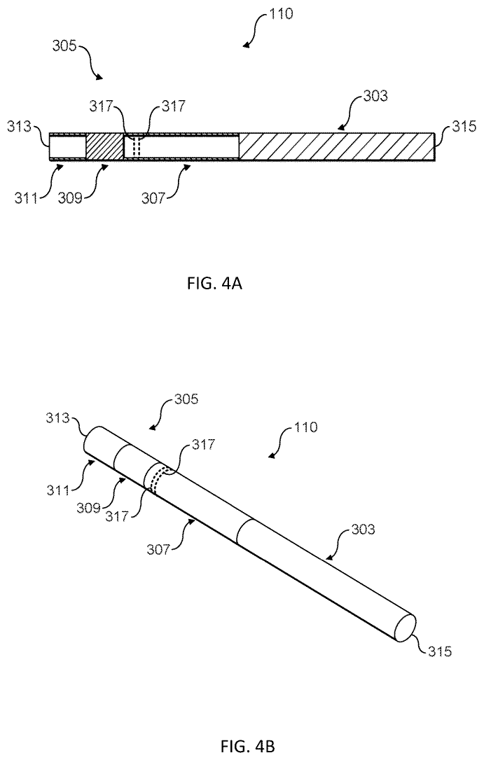

Referring to FIGS. 4A and 4B, there is shown a partially cut-away section view and a perspective view of an embodiment of an aerosol generating article 110 as disclosed herein. The article 110. In use, the article 110 is removably inserted into the device 100 shown in FIG. 1 at the opening 104 of the device 100.

The article 110 of one embodiment is in the form of a substantially cylindrical rod that includes a body of aerosol generating material 303 as disclosed herein and a filter assembly 305 in the form of a rod. The filter assembly 305 includes three segments, a cooling segment 307, a filter segment 309 and a mouth end segment 311. The article 110 has a first end 313, also known as a mouth end or a proximal end and a second end 315, also known as a distal end. The body of aerosol generating material 303 is located towards the distal end 315 of the article 110. In one example, the cooling segment 307 is located adjacent the body of aerosol generating material 303 between the body of aerosol generating material 303 and the filter segment 309, such that the cooling segment 307 is in an abutting relationship with the aerosol generating material 303 and the filter segment 309. In other examples, there may be a separation between the body of aerosol generating material 303 and the cooling segment 307 and between the body of aerosol generating material 303 and the filter segment 309. The filter segment 309 is located in between the cooling segment 307 and the mouth end segment 311. The mouth end segment 311 is located towards the proximal end 313 of the article 110, adjacent the filter segment 309. In one example, the filter segment 309 is in an abutting relationship with the mouth end segment 311. In one embodiment, the total length of the filter assembly 305 is between 37 mm and 45 mm, more preferably, the total length of the filter assembly 305 is 41 mm.

In one embodiment, the body of aerosol generating material 303 is between 10 mm and 100 mm in length, for instance between 10 mm and 15 mm in length, between 15 mm and 100 mm in length, between 34 mm and 50 mm in length, more preferably, the body of aerosol generating material 303 is between 38 mm and 46 mm in length, more preferably still, the body of aerosol generating material 303 is 42 mm in length.

In one embodiment, the total length of the article 110 is between 71 mm and 95 mm, more preferably, total length of the article 110 is between 79 mm and 87 mm, more preferably still, total length of the article 110 is 83 mm.

An axial end of the body of aerosol generating material 303 is visible at the distal end 315 of the article 110. However, in other embodiments, the distal end 315 of the article 110 may comprise an end member (not shown) covering the axial end of the body of aerosol generating material 303.

The body of aerosol generating material 303 is joined to the filter assembly 305 by annular tipping paper (not shown), which is located substantially around the circumference of the filter assembly 305 to surround the filter assembly 305 and extends partially along the length of the body of aerosol generating material 303. In one example, the tipping paper is made of 58 GSM standard tipping base paper. In one example has a length of between 42 mm and 50 mm, and more preferably, the tipping paper has a length of 46 mm.

In one embodiment, the cooling segment 307 is an annular tube and is located around and defines an air gap within the cooling segment. The air gap provides a chamber for heated volatilized components generated from the body of aerosol generating material 303 to flow. The cooling segment 307 is hollow to provide a chamber for aerosol accumulation yet rigid enough to withstand axial compressive forces and bending moments that might arise during manufacture and whilst the article 110 is in use during insertion into the device 100. In one embodiment, the thickness of the wall of the cooling segment 307 is approximately 0.29 mm.

The cooling segment 307 provides a physical displacement between the aerosol generating material 303 and the filter segment 309. The physical displacement provided by the cooling segment 307 will provide a thermal gradient across the length of the cooling segment 307. In one example the cooling segment 307 is configured to provide a temperature differential of at least 40 degrees Celsius between a heated volatilized component entering a first end of the cooling segment 307 and a heated volatilized component exiting a second end of the cooling segment 307. In one example the cooling segment 307 is configured to provide a temperature differential of at least 60 degrees Celsius, and more preferably at least 100 degrees Celsius between a heated volatilized component entering a first end of the cooling segment 307 and a heated volatilized component exiting a second end of the cooling segment 307. This temperature differential across the length of the cooling element 307 protects the temperature sensitive filter segment 309 from the high temperatures of the aerosol generating material 303 when it is heated by the heating arrangement of the device 100. If the physical displacement was not provided between the filter segment 309 and the body of aerosol generating material 303 and the heating elements of the device 100, then the temperature sensitive filter segment 309 may become damaged in use, so it would not perform its required functions as effectively.

In one embodiment, the length of the cooling segment 307 is at least 15 mm. In one embodiment, the length of the cooling segment 307 is between 20 mm and 30 mm, more particularly 23 mm to 27 mm, more particularly 25 mm to 27 mm and more particularly 25 mm.

The cooling segment 307 is made of paper, which means that it is comprised of a material that does not generate compounds of concern, for example, toxic compounds when in use adjacent to the heater arrangement of the device 100. In one example, the cooling segment 307 is manufactured from a spirally wound paper tube which provides a hollow internal chamber yet maintains mechanical rigidity. Spirally wound paper tubes are able to meet the tight dimensional accuracy requirements of high-speed manufacturing processes with respect to tube length, outer diameter, roundness and straightness.

In another embodiment, the cooling segment 307 is a recess created from stiff plug wrap or tipping paper. The stiff plug wrap or tipping paper is manufactured to have a rigidity that is sufficient to withstand the axial compressive forces and bending moments that might arise during manufacture and whilst the article 110 is in use during insertion into the device 100.

For each of the embodiments of the cooling segment 307, the dimensional accuracy of the cooling segment is sufficient to meet the dimensional accuracy requirements of high-speed manufacturing process.

The filter segment 309 may be formed of any filter material sufficient to remove one or more volatilized compounds from heated volatilized components from the aerosol generating material. In one example the filter segment 309 is made of a mono-acetate material, such as cellulose acetate. The filter segment 309 provides cooling and irritation-reduction from the heated volatilized components without depleting the quantity of the heated volatilized components to an unsatisfactory level for a user.

The density of the cellulose acetate tow material of the filter segment 309 controls the pressure drop across the filter segment 309, which in turn controls the draw resistance of the article 110. Therefore, the selection of the material of the filter segment 309 is important in controlling the resistance to draw of the article 110. In addition, the filter segment 309 performs a filtration function in the article 110.

In one embodiment, the filter segment 309 is made of an 8Y15 grade of filter tow material, which provides a filtration effect on the heated volatilized material, whilst also reducing the size of condensed aerosol droplets which result from the heated volatilized material which consequentially reduces the irritation and throat impact of the heated volatilized material to satisfactory levels.

The presence of the filter segment 309 provides an insulating effect by providing further cooling to the heated volatilized components that exit the cooling segment 307. This further cooling effect reduces the contact temperature of the user's lips on the surface of the filter segment 309.

An aerosol-modifying agent as disclosed herein may be added to the filter segment 309 in the form of either direct injection into the filter segment 309 or by embedding or arranging one or more breakable capsules (or other aerosol-modifying agent release component) as disclosed herein within the cellulose acetate tow of the filter segment 309.

In one example, the filter segment 309 is between 6 mm to 10 mm in length, more preferably 8 mm.

The mouth end segment 311 is an annular tube and is located around and defines an air gap within the mouth end segment 311. The air gap provides a chamber for heated volatilized components that flow from the filter segment 309. The mouth end segment 311 is hollow to provide a chamber for aerosol accumulation yet rigid enough to withstand axial compressive forces and bending moments that might arise during manufacture and whilst the article is in use during insertion into the device 100. In one example, the thickness of the wall of the mouth end segment 311 is approximately 0.29 mm.

In one embodiment, the length of the mouth end segment 311 is between 6 mm to 10 mm and more preferably 8 mm. In one example, the thickness of the mouth end segment is 0.29 mm.

The mouth end segment 311 may be manufactured from a spirally wound paper tube which provides a hollow internal chamber yet maintains critical mechanical rigidity. Spirally wound paper tubes are able to meet the tight dimensional accuracy requirements of high-speed manufacturing processes with respect to tube length, outer diameter, roundness and straightness.

The mouth end segment 311 provides the function of preventing any liquid condensate that accumulates at the exit of the filter segment 309 from coming into direct contact with a user.

It should be appreciated that, in one example, the mouth end segment 311 and the cooling segment 307 may be formed of a single tube and the filter segment 309 is located within that tube separating the mouth end segment 311 and the cooling segment 307.

A ventilation region 317 is provided in the article 110 to enable air to flow into the interior of the article 110 from the exterior of the article 110. In one example the ventilation region 317 takes the form of one or more ventilation holes 317 formed through the outer layer of the article 110. The ventilation holes may be located in the cooling segment 307 to aid with the cooling of the article 301. In one example, the ventilation region 317 comprises one or more rows of holes, and preferably, each row of holes is arranged circumferentially around the article 110 in a cross-section that is substantially perpendicular to a longitudinal axis of the article 110.

In one embodiment, there are between one to four rows of ventilation holes to provide ventilation for the article 110. Each row of ventilation holes may have between 12 to 36 ventilation holes 317. The ventilation holes 317 may, for example, be between 100 to 500 μm in diameter. In one example, an axial separation between rows of ventilation holes 317 is between 0.25 mm and 0.75 mm, more preferably, an axial separation between rows of ventilation holes 317 is 0.5 mm.

In one example, the ventilation holes 317 are of uniform size. In another example, the ventilation holes 317 vary in size. The ventilation holes can be made using any suitable technique, for example, one or more of the following techniques: laser technology, mechanical perforation of the cooling segment 307 or pre-perforation of the cooling segment 307 before it is formed into the article 110. The ventilation holes 317 are positioned so as to provide effective cooling to the article 110.

In one embodiment, the rows of ventilation holes 317 are located at least 11 mm from the proximal end 313 of the article, more preferably the ventilation holes are located between 17 mm and 20 mm from the proximal end 313 of the article 110. The location of the ventilation holes 317 is positioned such that user does not block the ventilation holes 317 when the article 110 is in use.

Advantageously, providing the rows of ventilation holes between 17 mm and 20 mm from the proximal end 313 of the article 110 enables the ventilation holes 317 to be located outside of the device 100, when the article 110 is fully inserted in the device 100, as can be seen in FIG. 1. By locating the ventilation holes outside of the apparatus, non-heated air is able to enter the article 110 through the ventilation holes from outside the device 100 to aid with the cooling of the article 110.

The length of the cooling segment 307 is such that the cooling segment 307 will be partially inserted into the device 100, when the article 110 is fully inserted into the device 100. The length of the cooling segment 307 provides a first function of providing a physical gap between the heater arrangement of the device 100 and the heat sensitive filter arrangement 309, and a second function of enabling the ventilation holes 317 to be located in the cooling segment, whilst also being located outside of the device 100, when the article 110 is fully inserted into the device 100. As can be seen from FIG. 1, the majority of the cooling element 307 is located within the device 100. However, there is a portion of the cooling element 307 that extends out of the device 100. It is in this portion of the cooling element 307 that extends out of the device 100 in which the ventilation holes 317 are located.

In the embodiment illustrated in FIGS. 4A and 4B, the article has a total length of 83 mm, including a 42 mm long cylindrical tobacco rod (diameter 5.4 mm) containing approximately 260 mg of aerosol generating material. The article has a ventilation ratio of 75%. The article is used in a device having a susceptor with a length of 44.5 mm and an internal diameter of 5.55 mm.

In another embodiment (not illustrated), the article has a total length of 75 mm, including a 34 mm long cylindrical tobacco rod (diameter 6.7 mm) containing approximately 340 mg of aerosol generating material. The article may have a ventilation ratio of 60%. This is used in a device having a susceptor with a length of 36 mm and an internal diameter of 7.1 mm.

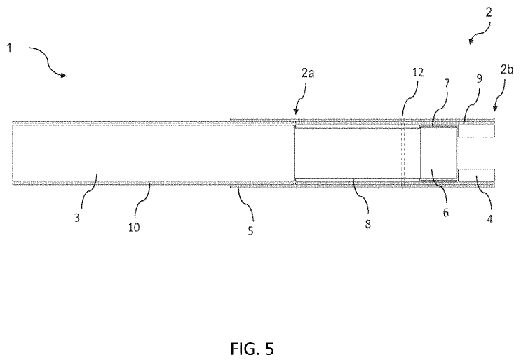

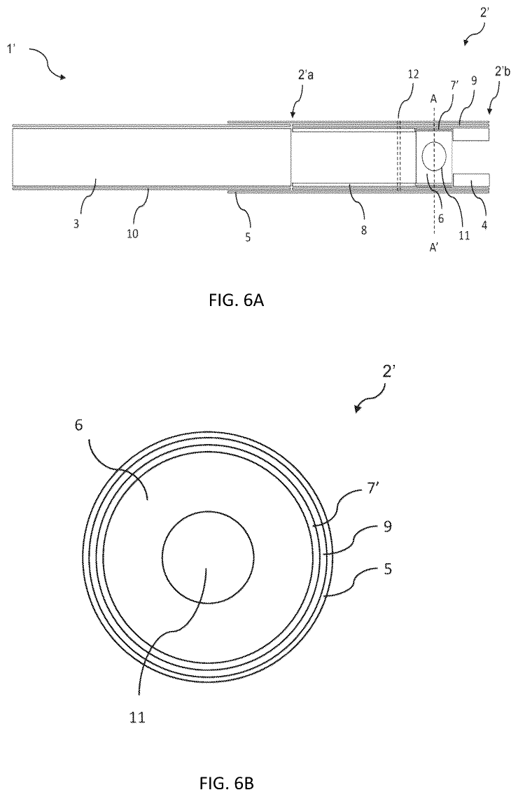

Further embodiments of an article are illustrated in FIGS. 5, 6A, and 6B. In some embodiments, the aerosol generating material contains an aerosol-modifying agent as described herein, e.g., in the form of capsules and/or adsorbed or absorbed in a wrapping element, are present in these example embodiments.

As shown in FIG. 5, the mouthpiece 2 of the article 1 comprises an upstream end 2a adjacent to the aerosol generating substrate 3 and a downstream end 2b distal from the aerosol generating substrate 3. At the downstream end 2b, the mouthpiece 2 has a hollow tubular element 4 formed from filamentary tow. This has advantageously been found to significantly reduce the temperature of the outer surface of the mouthpiece 2 at the downstream end 2b of the mouthpiece which comes into contact with a consumer's mouth when the article 1 is in use. In addition, the use of the tubular element 4 has also been found to significantly reduce the temperature of the outer surface of the mouthpiece 2 even upstream of the tubular element 4. Without wishing to be bound by theory, it is hypothesized that this is due to the tubular element 4 channeling aerosol closer to the center of the mouthpiece 2, and therefore reducing the transfer of heat from the aerosol to the outer surface of the mouthpiece 2.

In the present embodiment, the article 1 has an outer circumference of about 21 mm (i.e., the article is in the demi-slim format). In other examples, the article can be provided in any of the formats described herein, for instance having an outer circumference of between 15 mm and 25 mm. Since the article is to be heated to release an aerosol, improved heating efficiency can be achieved using articles having lower outer circumferences within this range, for instance circumferences of less than 23 mm. To achieve improved aerosol via heating, while maintaining a suitable product length, article circumferences of greater than 19 mm have also been found to be particularly effective. Articles having circumferences of between 19 mm and 23 mm, and more preferably between 20 mm and 22 mm, have been found to provide a good balance between providing effective aerosol delivery while allowing for efficient heating.

The outer circumference of the mouthpiece 2 is substantially the same as the outer circumference of the rod of aerosol generating material 3, such that there is a smooth transition between these components. In the present example, the outer circumference of the mouthpiece 2 is about 20.8 mm. A tipping paper 5 is wrapped around the full length of the mouthpiece 2 and over part of the rod of aerosol generating material 3 and has an adhesive on its inner surface to connect the mouthpiece 2 and rod 3. In the present example, the tipping paper 5 extends 5 mm over the rod of aerosol generating material 3 but it can alternatively extend between 3 mm and 10 mm over the rod 3, or more preferably between 4 mm and 6 mm, to provide a secure attachment between the mouthpiece 2 and rod 3. The tipping paper 5 can have a basis weight which is higher than the basis weight of plug wraps used in the article 1, for instance a basis weight of 40 gsm to 80 gsm, more preferably between 50 gsm and 70 gsm, and in the present example 58 gsm. These ranges of basis weights have been found to result in tipping papers having acceptable tensile strength while being flexible enough to wrap around the article 1 and adhere to itself along a longitudinal lap seam on the paper. The outer circumference of the tipping paper 5, once wrapped around the mouthpiece 2, is about 21 mm.

The “wall thickness” of the hollow tubular element 4 corresponds to the thickness of the wall of the tube 4 in a radial direction. This may be measured, for example, using a caliper. The wall thickness is advantageously greater than 0.9 mm, and more preferably 1.0 mm or greater. Preferably, the wall thickness is substantially constant around the entire wall of the hollow tubular element 4. However, where the wall thickness is not substantially constant, the wall thickness is preferably greater than 0.9 mm at any point around the hollow tubular element 4, more preferably 1.0 mm or greater.

Preferably, the length of the hollow tubular element 4 is less than about 20 mm. More preferably, the length of the hollow tubular element 4 is less than about 15 mm. Still more preferably, the length of the hollow tubular element 4 is less than about 10 mm. In addition, or as an alternative, the length of the hollow tubular element 4 is at least about 5 mm. Preferably, the length of the hollow tubular element 4 is at least about 6 mm. In some preferred embodiments, the length of the hollow tubular element 4 is from about 5 mm to about 20 mm, more preferably from about 6 mm to about 10 mm, even more preferably from about 6 mm to about 8 mm, most preferably about 6 mm, 7 mm or about 8 mm. In the present example, the length of the hollow tubular element 4 is 6 mm.

Preferably, the density of the hollow tubular element 4 is at least about 0.25 grams per cubic centimeter (g/cc), more preferably at least about 0.3 g/cc. Preferably, the density of the hollow tubular element 4 is less than about 0.75 grams per cubic centimeter (g/cc), more preferably less than 0.6 g/cc. In some embodiments, the density of the hollow tubular element 4 is between 0.25 and 0.75 g/cc, more preferably between 0.3 and 0.6 g/cc, and more preferably between 0.4 g/cc and 0.6 g/cc or about 0.5 g/cc. These densities have been found to provide a good balance between improved firmness afforded by denser material and the lower heat transfer properties of lower density material. For the purposes of the present disclosure, the “density” of the hollow tubular element 4 refers to the density of the filamentary tow forming the element with any plasticizer incorporated. The density may be determined by dividing the total weight of the hollow tubular element 4 by the total volume of the hollow tubular element 4, wherein the total volume can be calculated using appropriate measurements of the hollow tubular element 4 taken, for example, using calipers. Where necessary, the appropriate dimensions may be measured using a microscope.

The filamentary tow forming the hollow tubular element 4 preferably has a total denier of less than 45,000, more preferably less than 42,000. This total denier has been found to allow the formation of a tubular element 4 which is not too dense. Preferably, the total denier is at least 20,000, more preferably at least 25,000. In preferred embodiments, the filamentary tow forming the hollow tubular element 4 has a total denier between 25,000 and 45,000, more preferably between 35,000 and 45,000. Preferably the cross-sectional shape of the filaments of tow are ‘Y’ shaped, although in other embodiments other shapes such as ‘X’ shaped filaments can be used.

The filamentary tow forming the hollow tubular element 4 preferably has a denier per filament of greater than 3. This denier per filament has been found to allow the formation of a tubular element 4 which is not too dense. Preferably, the denier per filament is at least 4, more preferably at least 5. In preferred embodiments, the filamentary tow forming the hollow tubular element 4 has a denier per filament between 4 and 10, more preferably between 4 and 9. In one example, the filamentary tow forming the hollow tubular element 4 has an 8Y40,000 tow formed from cellulose acetate and comprising 18% plasticizer, for instance triacetin.

The hollow tubular element 4 preferably has an internal diameter of greater than 3.0 mm. Smaller diameters than this can result in increasing the velocity of aerosol passing though the mouthpiece 2 to the consumers mouth more than is desirable, such that the aerosol becomes too warm, for instance reaching temperatures greater than 40° C. or greater than 45° C. More preferably, the hollow tubular element 4 has an internal diameter of greater than 3.1 mm, and still more preferably greater than 3.5 mm or 3.6 mm. In one embodiment, the internal diameter of the hollow tubular element 4 is about 3.9 mm.

The hollow tubular element 4 preferably comprises from 15% to 22% by weight of plasticizer. For cellulose acetate tow, the plasticizer is preferably triacetin, although other plasticizers such as polyethylene glycol (PEG) can be used. More preferably, the tubular element 4 comprises from 16% to 20% by weight of plasticizer, for instance about 17%, about 18% or about 19% plasticizer.

The pressure drop or difference (also referred to a resistance to draw) across the mouthpiece, for instance the part of the article 1 downstream of the aerosol generating material 3, is preferably less than about 40 mm H₂O. Such pressure drops have been found to allow sufficient aerosol, including desirable compounds such as flavor compounds, to pass through the mouthpiece 2 to the consumer. More preferably, the pressure drop across the mouthpiece 2 is less than about 32 mm H₂O. In some embodiments, particularly improved aerosol has been achieved using a mouthpiece 2 having a pressure drop of less than 31 mm H₂O, for instance about 29 mm H₂O, about 28 mm H₂O or about 27.5 mm H₂O. Alternatively or additionally, the mouthpiece pressure drop can be at least 10 mm H₂O, preferably at least 15 mm H₂O and more preferably at least 20 mm H₂O. In some embodiments, the mouthpiece pressure drop can be between about 15 mm H₂O and 40 mm H₂O. These values enable the mouthpiece 2 to slow down the aerosol as it passes through the mouthpiece 2 such that the temperature of the aerosol has time to reduce before reaching the downstream end 2b of the mouthpiece 2.

The mouthpiece 2, in the present example, includes a body of material 6 upstream of the hollow tubular element 4, in this example adjacent to and in an abutting relationship with the hollow tubular element 4. The body of material 6 and hollow tubular element 4 each define a substantially cylindrical overall outer shape and share a common longitudinal axis. The body of material 6 is wrapped in a first plug wrap 7. Preferably, the first plug wrap 7 has a basis weight of less than 50 gsm, more preferably between about 20 gsm and 40 gsm. Preferably, the first plug wrap 7 has a thickness of between 30 μm and 60 μm, more preferably between 35 μm and 45 μm. Preferably, the first plug wrap 7 is a non-porous plug wrap, for instance having a permeability of less than 100 Coresta units, for instance less than 50 Coresta units. However, in other embodiments, the first plug wrap 7 can be a porous plug wrap, for instance having a permeability of greater than 200 Coresta Units.

Preferably, the length of the body of material 6 is less than about 15 mm. More preferably, the length of the body of material 6 is less than about 10 mm. In addition, or as an alternative, the length of the body of material 6 is at least about 5 mm. Preferably, the length of the body of material 6 is at least about 6 mm. In some preferred embodiments, the length of the body of material 6 is from about 5 mm to about 15 mm, more preferably from about 6 mm to about 12 mm, even more preferably from about 6 mm to about 12 mm, most preferably about 6 mm, 7 mm, 8 mm, 9 mm or 10 mm. In the present example, the length of the body of material 6 is 10 mm.

In the present embodiment, the body of material 6 is formed from filamentary tow. In the present example, the tow used in the body of material 6 has a denier per filament (d.p.f) of 8.4 and a total denier of 21,000. Alternatively, the tow can, for instance, have a denier per filament (d.p.f) of 9.5 and a total denier of 12,000. In the present example, the tow comprises plasticized cellulose acetate tow. The plasticizer used in the tow comprises about 7% by weight of the tow. In the present example, the plasticizer is triacetin. In other examples, different materials can be used to form the body of material 6. For instance, rather than tow, the body 6 can be formed from paper, for instance in a similar way to paper filters known for use in cigarettes. Alternatively, the body 6 can be formed from tows other than cellulose acetate, for instance polylactic acid (PLA), other materials described herein for filamentary tow or similar materials. The tow is preferably formed from cellulose acetate. The tow, whether formed from cellulose acetate or other materials, preferably has a d.p.f of at least 5, more preferably at least 6 and still more preferably at least 7. These values of denier per filament provide a tow which has relatively coarse, thick fibers with a lower surface area which result in a lower pressure drop across the mouthpiece 2 than tows having lower d.p.f values. Preferably, to achieve a sufficiently uniform body of material 6, the tow has a denier per filament of no more than 12 d.p.f, preferably no more than 11 d.p.f and still more preferably no more than 10 d.p.f.

The total denier of the tow forming the body of material 6 is preferably at most 30,000, more preferably at most 28,000 and still more preferably at most 25,000. These values of total denier provide a tow which takes up a reduced proportion of the cross-sectional area of the mouthpiece 2 which results in a lower pressure drop across the mouthpiece 2 than tows having higher total denier values. For appropriate firmness of the body of material 6, the tow preferably has a total denier of at least 8,000 and more preferably at least 10,000. Preferably, the denier per filament is between 5 and 12 while the total denier is between 10,000 and 25,000. More preferably, the denier per filament is between 6 and 10 while the total denier is between 11,000 and 22,000. Preferably the cross-sectional shape of the filaments of tow are ‘Y’ shaped, although in other embodiments other shapes such as ‘X’ shaped filaments can be used, with the same d.p.f and total denier values as provided herein.

In the present embodiment, the hollow tubular element 4 is a first hollow tubular element 4 and the mouthpiece includes a second hollow tubular element 8 upstream of the first hollow tubular element 4. In the present example, the second hollow tubular element 8 is upstream of, adjacent to and in an abutting relationship with the body of material 6. The body of material 6 and second hollow tubular element 8 each define a substantially cylindrical overall outer shape and share a common longitudinal axis. The second hollow tubular element 8 is formed from a plurality of layers of paper which are parallel wound, with butted seams, to form the tubular element 8. In the present example, first and second paper layers are provided in a two-ply tube, although in other examples 3, 4 or more paper layers can be used forming 3, 4 or more ply tubes. Other constructions can be used, such as spirally wound layers of paper, cardboard tubes, tubes formed using a papier-mâché type process, molded or extruded plastic tubes or similar. The second hollow tubular element 8 can also be formed using a stiff plug wrap and/or tipping paper as the second plug wrap 9 and/or tipping paper 5 described herein, meaning that a separate tubular element is not required. The stiff plug wrap and/or tipping paper is manufactured to have a rigidity that is sufficient to withstand the axial compressive forces and bending moments that might arise during manufacture and whilst the article 1 is in use. For instance, the stiff plug wrap and/or tipping paper can have a basis weight between 70 gsm and 120 gsm, more preferably between 80 gsm and 110 gsm. Additionally or alternatively, the stiff plug wrap and/or tipping paper can have a thickness between 80 μm and 200 μm, more preferably between 100 μm and 160 μm, or from 120 μm to 150 μm. It can be desirable for both the second plug wrap 9 and tipping paper 5 to have values in these ranges, to achieve an acceptable overall level of rigidity for the second hollow tubular element 8.

The second hollow tubular element 8 preferably has a wall thickness, which can be measured in the same way as that of the first hollow tubular element 4, of at least about 100 μm and up to about 1.5 mm, preferably between 100 μm and 1 mm and more preferably between 150 μm and 500 μm, or about 300 μm. In the present example, the second hollow tubular element 8 has a wall thickness of about 290 μm.

Preferably, the length of the second hollow tubular element 8 is less than about 50 mm. More preferably, the length of the second hollow tubular element 8 is less than about 40 mm. Still more preferably, the length of the second hollow tubular element 8 is less than about 30 mm. In addition, or as an alternative, the length of the second hollow tubular element 8 is preferably at least about 10 mm. Preferably, the length of the second hollow tubular element 8 is at least about 15 mm. In some preferred embodiments, the length of the second hollow tubular element 8 is from about 20 mm to about 30 mm, more preferably from about 22 mm to about 28 mm, even more preferably from about 24 to about 26 mm, most preferably about 25 mm. In the present example, the length of the second hollow tubular element 8 is 25 mm.

The second hollow tubular element 8 is located around and defines an air gap within the mouthpiece 2 which acts as a cooling segment. The air gap provides a chamber through which heated volatilized components generated by the aerosol generating material 3 flow. The second hollow tubular element 8 is hollow to provide a chamber for aerosol accumulation yet rigid enough to withstand axial compressive forces and bending moments that might arise during manufacture and whilst the article 1 is in use. The second hollow tubular element 8 provides a physical displacement between the aerosol generating material 3 and the body of material 6. The physical displacement provided by the second hollow tubular element 8 will provide a thermal gradient across the length of the second hollow tubular element 8.

Preferably, the mouthpiece 2 comprises a cavity having an internal volume greater than 450 mm³. Providing a cavity of at least this volume has been found to enable the formation of an improved aerosol. Such a cavity size provides sufficient space within the mouthpiece 2 to allow heated volatilized components to cool, therefore allowing the exposure of the aerosol generating material 3 to higher temperatures than would otherwise be possible, since they may result in an aerosol which is too warm. In the present example, the cavity is formed by the second hollow tubular element 8, but in alternative arrangements it could be formed within a different part of the mouthpiece 2. More preferably, the mouthpiece 2 comprises a cavity, for instance formed within the second hollow tubular element 8, having an internal volume greater than 500 mm³, and still more preferably greater than 550 mm³, allowing further improvement of the aerosol. In some examples, the internal cavity comprises a volume of between about 550 mm³ and about 750 mm³, for instance about 600 mm³ or 700 mm³.

The second hollow tubular element 8 has a similar function to the cooling segment 307 as described above and has similar advantages as described herein.

In the present example, the first hollow tubular element 4, body of material 6 and second hollow tubular element 8 are combined using a second plug wrap 9 which is wrapped around all three sections. Preferably, the second plug wrap 9 has a basis weight of less than 50 gsm, more preferably between about 20 gsm and 45 gsm. Preferably, the second plug wrap 9 has a thickness of between 30 μm and 60 μm, more preferably between 35 μm and 45 μm. The second plug wrap 9 is preferably a non-porous plug wrap having a permeability of less than 100 Coresta Units, for instance less than 50 Coresta Units. However, in alternative embodiments, the second plug wrap 9 can be a porous plug wrap, for instance having a permeability of greater than 200 Coresta Units.

In the present example, the aerosol generating material 3 is wrapped in a wrapper 10. The wrapper 10 can, for instance, be a paper or paper-backed foil wrapper. In the present example, the wrapper 10 is substantially impermeable to air. In alternative embodiments, the wrapper 10 preferably has a permeability of less than 100 Coresta Units, more preferably less than 60 Coresta Units. It has been found that low permeability wrappers, for instance having a permeability of less than 100 Coresta Units, more preferably less than 60 Coresta Units, result in an improvement in the aerosol formation in the aerosol generating material 3. Without wishing to be bound by theory, it is hypothesized that this is due to reduced loss of aerosol compounds through the wrapper 10. The permeability of the wrapper 10 can be measured in accordance with ISO 2965:2009 concerning the determination of air permeability for materials used as cigarette papers, filter plug wrap and filter joining paper.

In the present embodiment, the wrapper 10 comprises aluminum foil. Aluminum foil has been found to be particularly effective at enhancing the formation of aerosol within the aerosol generating material 3. In the present example, the aluminum foil has a metal layer having a thickness of about 6 μm. In the present example, the aluminum foil has a paper backing. However, in alternative arrangements, the aluminum foil can be other thicknesses, for instance between 4 μm and 16 μm in thickness. The aluminum foil also need not have a paper backing, but could have a backing formed from other materials, for instance to help provide an appropriate tensile strength to the foil, or it could have no backing material. Metallic layers or foils other than aluminum can also be used. The total thickness of the wrapper is preferably between 20 μm and 60 μm, more preferably between 30 μm and 50 μm, which can provide a wrapper having appropriate structural integrity and heat transfer characteristics. The tensile force which can be applied to the wrapper before it breaks can be greater than 3,000 grams force, for instance between 3,000 and 10,000 grams force or between 3,000 and 4,500 grams force.

The article 1 has a ventilation level of about 75% of the aerosol drawn through the article. In alternative embodiments, the article can have a ventilation level of between 50% and 80% of aerosol drawn through the article, for instance between 65% and 75%. Ventilation at these levels helps to slow down the flow of aerosol drawn through the mouthpiece 2 and thereby enable the aerosol to cool sufficiently before it reaches the downstream end 2 b of the mouthpiece 2. The ventilation is provided directly into the mouthpiece 2 of the article 1. In the present example, the ventilation is provided into the second hollow tubular element 8, which has been found to be particularly beneficial in assisting with the aerosol generation process. The ventilation is provided via first and second parallel rows of perforations 12, in the present case formed as laser perforations, at positions 17.925 mm and 18.625 mm respectively from the downstream, mouth-end 2 b of the mouthpiece 2. These perforations pass though the tipping paper 5, second plug wrap 9 and second hollow tubular element 8. In alternative embodiments, the ventilation can be provided into the mouthpiece at other locations, for instance into the body of material 6 or first tubular element 4.

In some embodiments, the aerosol forming material added to the aerosol generating material 3 comprises about 14% by weight of the aerosol generating material 3. In some embodiments, the aerosol forming material comprises at least 5% by weight of the aerosol generating material, such as at least 10%. In some embodiments, the aerosol forming material comprises less than 25% by weight of the aerosol generating material, such as less than 20%, for instance between 10% and 20%, between 12% and 18% or between 13% and 16%.

In some embodiments, the aerosol generating material 3 is provided as a cylindrical rod. Irrespective of the form of the aerosol generating material, it preferably has a length of about 10 mm to 100 mm. In some embodiments, the length of the aerosol generating material is in the range about 25 mm to 50 mm, such as in the range about 30 mm to 45 mm, or about 30 mm to 40 mm.

The volume of aerosol generating material 3 provided can vary from about 200 mm³ to about 4300 mm³, such as from about 500 mm³ to 1500 mm³, or from about 1000 mm³ to about 1300 mm³. The provision of these volumes of aerosol generating material, for instance from about 1000 mm³ to about 1300 mm³, has been advantageously shown to achieve a superior aerosol, having a greater visibility and sensory performance compared to that achieved with volumes selected from the lower end of the range.

The mass of aerosol generating material 3 provided can be greater than 200 mg, for instance from about 200 mg to 400 mg, such as from about 230 mg to 360 mg, or from about 250 mg to 360 mg.

In some embodiments, the article 1 contains an aerosol-modifying agent as described herein, e.g., in the form of capsules (or other aerosol-modifying agent release component) contained within any component of the article and/or adsorbed or absorbed in a wrapping element.

FIG. 6A is a side-on cross-sectional view of a further article 1′ including a capsule-containing mouthpiece 2′, the capsule comprising an aerosol-modifying agent as described herein. FIG. 6B is a cross sectional view of the capsule-containing mouthpiece shown in FIG. 6A through the line A-A′ thereof. Article 1′ and capsule-containing mouthpiece 2′ are the same as the article 1 and mouthpiece 2 illustrated in FIG. 5, except that an aerosol-modifying agent as disclosed herein is provided within the body of material 6, in the present example in the form of a capsule 11, and that an oil-resistant first plug wrap 7′ surrounds the body of material 6. In other examples, the aerosol-modifying agent can be provided in other forms, such as material injected into the body of material 6 or provided on a thread, which may also be disposed within the body of material 6. Use of the capsule 11 can advantageously retain the aerosol-modifying agent until use of the article 1′ by the user, which can enhance shelf life of the aerosol-modifying agent. The user can, for example, apply pressure to the article 1′ in the area of the capsule 11 to cause rupture of the capsule and release of the aerosol-modifying agent.

In the present embodiment, a single capsule 11 is used. The capsule 11 is entirely embedded within the body of material 6. In other words, the capsule 11 is completely surrounded by the material forming the body 6. In other embodiments, a plurality of breakable capsules may be disposed within the body of material 6, for instance 2, 3 or more breakable capsules. The length of the body of material 6 can be increased to accommodate the number of capsules required. In examples where a plurality of capsules is used, the individual capsules may be the same as each other, or may differ from one another in terms of size and/or capsule payload. In other examples, multiple bodies of material 6 may be provided, with each body containing one or more capsules.

Although the illustrated embodiment of FIG. 6A and FIG. 6B show the capsule 11 within the body 6, the capsule could be placed in other locations within the article 1′, such as within the tubular element 8 or within aerosol generating material 3.

The first plug wrap 7′ can comprise a barrier coating to make the material of the plug wrap substantially impermeable to the liquid payload of the capsule 11. Alternatively or in addition, the second plug wrap 9 and/or tipping paper 5 can comprise a barrier coating to make the material of that plug wrap and/or tipping paper substantially impermeable to the liquid payload of the capsule 11.

In the present embodiment, the capsule 11 is located at a longitudinally central position within the body of material 6. That is, the capsule 11 is positioned so that its center is 4 mm from each end of the body of material 6. In other embodiments, the capsule 11 can be located at a position other than a longitudinally central position in the body of material 6, i.e., closer to the downstream end of the body of material 6 than the upstream end, or closer to the upstream end of the body of material 6 than the downstream end. In some embodiments, the mouthpiece 2′ is configured so that the capsule 11 and the ventilation holes 12 are longitudinally offset from each other in the mouthpiece 2′.

A cross section of the mouthpiece 2′ is shown in FIG. 6B, this being taken through line A-A′ of FIG. 6A. FIG. 6B shows the capsule 11, the body of material 6, the first and second plug wraps 7′, 9 and the tipping paper 5. In the present example, the capsule 11 is centered on the longitudinal axis (not shown) of the mouthpiece 2′. The first and second plug wraps 7′, 9 and tipping 5 are arranged concentrically around the body of material 6.

The cross-sectional area of the capsule 11 at its largest cross-sectional area is in some embodiments less than 28% of the cross-sectional area of the portion of the mouthpiece 2′ in which the capsule 11 is provided, more preferably less than 27% and still more preferably less than 25%. For instance, for a spherical capsule having a diameter of 3.0 mm, the largest cross-sectional area of the capsule is 7.07 mm². For the mouthpiece 2′ having a circumference of 21 mm as described herein, the body of material 6 has an outer circumference of 20.8 mm, and the radius of this component will be 3.31 mm, corresponding to a cross sectional area of 34.43 mm². The capsule cross-sectional area is, in this example, 20.5% of the cross-sectional area of the mouthpiece 2′. As another example, if the capsule had a diameter of 3.2 mm, its largest cross-sectional area would be 8.04 mm². In this case, the cross-sectional area of the capsule would be 23.4% of the cross-sectional area of the body of material 6. A capsule with a largest cross sectional area less than 28% of the cross sectional area of the portion of the mouthpiece 2′ in which the capsule 11 is provided has the advantage that the pressure drop across the mouthpiece 2′ is reduced as compared to capsules with larger cross sectional areas and adequate space remains around the capsule for aerosol to pass without the body of material 6 removing significant amounts of the aerosol mass as it passes through the mouthpiece 2′.

Preferably the pressure drop or difference (also referred to a resistance to draw) across the article, measured as the open pressure drop (i.e. with the ventilation openings open), reduces by less than 8 mm H₂O when the capsule is broken. More preferably, the open pressure drop reduces by less than 6 mm H₂O and more preferably less than 5 mm H₂O. These values are measured as the average achieved by at least 80 articles made to the same design. Such small changes in pressure drop mean that other aspects of the product design, such as setting the correct ventilation level for a given product pressure drop, can be achieved irrespective of whether or not the consumer chooses to break the capsule.

In the examples described above, the mouthpieces 2, 2′ each comprise a single body of material 6. In other examples, either the mouthpiece of FIG. 5 or of FIGS. 6A and 6B may include multiple bodies of material. The mouthpieces 2, 2′ may comprise a cavity between the bodies of material.

In some examples, the mouthpiece 2, 2′ downstream of the aerosol generating material 3 can comprise a wrapper, for instance the first or second plug wraps 7, 9, or tipping paper 5, which comprises an aerosol-modifying agent as described herein. The aerosol-modifying agent may be disposed on an inwardly or outwardly facing surface of the mouthpiece wrapper. For instance, the aerosol-modifying agent may be provided on an area of the wrapper, such as an outwardly facing surface of the tipping paper 5, which comes into contact with the consumer's lips during use. By disposing the aerosol-modifying agent on the outwardly facing surface of the mouthpiece wrapper, the aerosol-modifying agent may be transferred to the consumer's lips during use. Transfer of the aerosol-modifying agent to the consumer's lips during use of the article may modify the organoleptic properties of the aerosol generated by the aerosol generating substrate 3 or otherwise provide the consumer with an alternative sensory experience. The aerosol-modifying agent may be at least partially soluble in water such that it is transferred to the user via the consumer's saliva. The aerosol-modifying agent may be one that volatilizes by the heat generated by the aerosol provision system. This may facilitate transfer of the aerosol-modifying agent to the aerosol generated by the aerosol generating substrate 3.

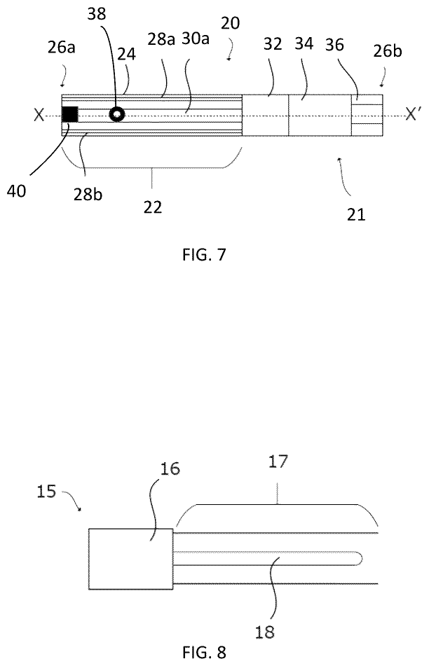

FIG. 7 illustrates a cross-sectional view of a further example of an article 20 for use in an aerosol delivery system. The article 20 comprises a mouthpiece 21, and an aerosol-generating section 22 connected to the mouthpiece 21. In the present example, the aerosol-generating section 22 comprises a body of aerosol-generating material 24 in the form of a rod. The article comprises a downstream end 26b and an upstream end 26a distal from the downstream end 26b. The article 20 comprises a longitudinal axis X-X′. The aerosol-generating section 22 comprises channels 28a, 28b and a cavity 30a for receiving an aerosol generator, as described below. A capsule 38 containing an aerosol-modifying agent is within the aerosol-generating section 22. A plug 40 can be placed at the upstream end 26a of the article 20 within the cavity 30a to retain the capsule 38 within the cavity. The plug 40 can be formed of any material capable of being pierced by, for example, an aerosol generator as discussed below. Example materials for plug 40 can include paper and filamentary tow.

The mouthpiece 21 includes a cooling section 32, also referred to as a cooling element, positioned immediately downstream of and adjacent to the aerosol-generating section 22. In the present example, the cooling section 32 is in an abutting relationship with the aerosol-generating material 24. The mouthpiece 21 also includes, in the present example, a body of material 34 downstream of the cooling section 32, and a hollow tubular element 36 downstream of the body of material 34, at the mouth end of the article 20.

The cooling section 32 is located defines an air gap within the mouthpiece 21 which acts as a cooling section. The air gap provides a chamber through which heated volatilized components generated by the aerosol-generating section 22. The cooling section 32 is hollow to provide a chamber for aerosol accumulation yet rigid enough to withstand axial compressive forces and bending moments that might arise during manufacture and whilst the article 20 is in use. The cooling section 32 provides a physical displacement between the aerosol-generating section 22 and the body of material 34. The physical displacement provided by the cooling section 32 can provide a thermal gradient across the length of the cooling section 32.

A tipping paper (not shown) is wrapped around the full length of the mouthpiece 21 and over part of the aerosol-generating section 22 and has an adhesive on its inner surface to connect the mouthpiece 21 and aerosol-generating section 22. In the present example, the aerosol-generating section 22 comprises a wrapper (not shown), which forms a first wrapping material, and the tipping paper forms an outer wrapping material which extends at least partially over the body of aerosol-generating material 24 to connect the mouthpiece 21 and the body of aerosol-generating material. In some examples, the tipping paper can extend only partially over the rod of aerosol-generating material.

In the present embodiment, a moisture impermeable wrapper (not shown) circumscribes aerosol-generating material 24 and comprises a paper wrapper. In other embodiments, the wrapper comprises an aluminum foil, optionally comprising a barrier coating to make the material of the wrapper substantially moisture impermeable.

Aluminum foil has been found to be particularly effective at enhancing the formation of aerosol within the aerosol-generating material. In the present example, the aluminum foil has a metal layer having a thickness of about 6 μm. In the present example, the aluminum foil has a paper backing. However, in alternative arrangements, the aluminum foil can be other thicknesses, for instance between 4 μm and 16 μm in thickness. The aluminum foil also need not have a paper backing, but could have a backing formed from other materials, for instance to help provide an appropriate tensile strength to the foil, or it could have no backing material. Metallic layers or foils other than aluminum can also be used. The total thickness of the wrapper is preferably between 20 μm and 60 μm, more preferably between 30 μm and 50 μm, which can provide a wrapper having appropriate structural integrity and heat transfer characteristics. The tensile force which can be applied to the wrapper before it breaks can be greater than 3,000 grams force, for instance between 3,000 and 10,000 grams force or between 3,000 and 4,500 grams force. Where the wrapper comprises paper or a paper backing, i.e. a cellulose based material, the wrapper can have a basis weight greater than about 30 gsm. For example, the wrapper can have a basis weight in the range from about 40 gsm to about 70 gsm. Such basis weights provide an improved rigidity to the rod of aerosol-generating material. The improved rigidity provided by wrappers having a basis weight in this range can make aerosol-generating section 22 more resistant to crumpling or other deformation under the forces to which the article is subject, in use.

In the present example, the moisture impermeable wrapper is also substantially impermeable to air. In alternative embodiments, the wrapper preferably has a permeability of less than 100 Coresta Units, more preferably less than 60 Coresta Units. It has been found that low permeability wrappers, for instance having a permeability of less than 100 Coresta Units, more preferably less than 60 Coresta Units, result in an improvement in the aerosol formation in the aerosol-generating section 11. Without wishing to be bound by theory, it is hypothesized that this is due to reduced loss of aerosol compounds through the wrapper. The permeability of the wrapper can be measured in accordance with ISO 2965:2009 concerning the determination of air permeability for materials used as cigarette papers, filter plug wrap and filter joining paper.

The body of material 34 and hollow tubular element 36 each define a substantially cylindrical overall outer shape and share a common longitudinal axis. The body of material 34 is wrapped in a first plug wrap (not shown). Preferably, the first plug wrap has a basis weight of less than 50 gsm, more preferably between about 20 gsm and 40 gsm. Preferably, the first plug wrap has a thickness of between 30 μm and 60 μm, more preferably between 35 μm and 45 μm. Preferably, the first plug wrap is a non-porous plug wrap, for instance having a permeability of less than 100 Coresta units, for instance less than 50 Coresta units. However, in other embodiments, the first plug wrap can be a porous plug wrap, for instance having a permeability of greater than 200 Coresta Units.

In the present example, the body of material 34 is formed from filamentary tow. In the present example, the tow used in the body of material 34 has a denier per filament (d.p.f.) of 8.4 and a total denier of 21,000. Alternatively, the tow can, for instance, have a denier per filament (d.p.f.) of 9.5 and a total denier of 12,000. In the present example, the tow comprises plasticized cellulose acetate tow. The plasticizer used in the tow comprises about 7% by weight of the tow. In the present example, the plasticizer is triacetin. In other examples, different materials can be used to form the body of material 34. For instance, rather than tow, the body 34 can be formed from paper, for instance in a similar way to paper filters known for use in cigarettes.

The article may have a ventilation level of about 10% of the aerosol drawn through the article. In alternative embodiments, the article can have a ventilation level of between 1% and 20% of aerosol drawn through the article, for instance between 1% and 12%. Ventilation at these levels helps to increase the consistency of the aerosol inhaled by the user at the mouth end 26b, while assisting the aerosol cooling process. The ventilation is provided directly into the mouthpiece 21 of the article 20. In the present example, the ventilation is provided into the cooling section 32, which has been found to be particularly beneficial in assisting with the aerosol generation process. The ventilation is provided via perforations, in the present case formed as a single row of laser perforations, positioned 13 mm from the downstream, mouth-end 26b of the mouthpiece 21. In alternative embodiments, two or more rows of ventilation perforations may be provided. These perforations pass though the tipping paper, second plug wrap and cooling section 32. In alternative embodiments, the ventilation can be provided into the mouthpiece at other locations, for instance into the body of material 34 or tubular element 36. Preferably, the article is configured such that the perforations are provided about 28 mm or less from the upstream end of the article 20, preferably between 20 mm and 28 mm from the upstream end of the article 20. In the present example, the apertures are provided about 25 mm from the upstream end of the article.

As shown FIG. 8, the components of an embodiment of a non-combustible aerosol provision device 15 are shown in a simplified manner. Particularly, the elements of the non-combustible aerosol provision device 15 are not drawn to scale in FIG. 8. Elements that are not relevant for the understanding of this embodiment have been omitted to simplify FIG. 8.

The non-combustible aerosol provision device 15 comprises a housing 16 comprising an area 17 for receiving an article. The area is in the form of a cavity, open at the proximal end (or mouth end) for receiving an aerosol-generating article, such as the article 20 of FIG. 7. The distal end of the cavity is spanned by an aerosol-generating assembly comprising an aerosol generator 18. In this embodiment, the aerosol-generator is in the form of a resistively-heated elongate pin.

The aerosol generator 18 is retained by a heater mount (not shown) such that an active heating area of the aerosol generator is located within the cavity. The active heating area of the aerosol generator 18 is positioned within the aerosol-generating section of an aerosol-generating article when the aerosol-generating article is fully received within the cavity.

The aerosol generator 18 is configured for insertion into the aerosol-generating section of an aerosol-generating article. As noted above, the aerosol generator 18 is shaped in the form of a pin terminating in a rounded point. The pin has a length dimension that is greater than its width dimension.

Use of the non-combustible aerosol provision device 15 with the article 20 of FIG. 7 will result in entry of the aerosol generator 18 into the cavity 30a at the upstream end 26a. The aerosol generator 18 is adapted to penetrate the plug 40 and the capsule 38, which will result in release of the aerosol-modifying agent from the capsule. In this manner, the capsule 38 can advantageously retain the aerosol-modifying agent until use of the article 20 by the user, which can enhance shelf life of the aerosol-modifying agent.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

1. An aerosol generating article comprising an aerosol generating material and an aerosol-modifying agent, wherein the aerosol-modifying agent comprises a compound having a structure according to Formula I, Formula II, or Formula III: 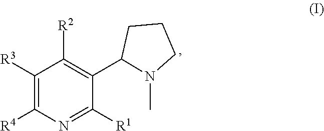
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or
the substituted 3-(1-methylpyrrolidin-2-yl)pyridine has a structure according to Formula II: 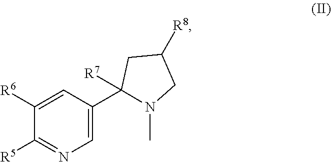
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C1-C3 alkyl; and
at least one of R⁷ and R⁸ is not hydrogen; or 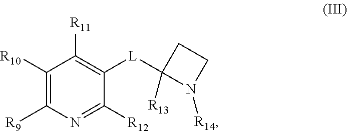
wherein
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The aerosol generating article of claim 1, aerosol-modifying agent comprises a compound having a structure according to Formula I, and wherein R¹, R², and R³ are each H.

3. The aerosol generating article of claim 2, wherein R⁴ is optionally substituted C₁-C₆ alkyl, F, Cl, Br, OCH₃, OEt, or CN.

4. The aerosol generating article of claim 3, wherein R⁴ is C₁-C₃ alkyl, such as wherein R⁴ is CH₃.

5. The aerosol generating article of claim 1, aerosol-modifying agent comprises a compound having a structure according to Formula II, wherein R⁵ and R⁶ are H; R⁷ is CH₃; and R⁸ is H.

6. The aerosol generating article of claim 1, aerosol-modifying agent comprises a compound having a structure according to Formula II, wherein R⁵, R⁶ and R⁷ are H, and R⁸ is CH₃.

7. The aerosol generating article of claim 1, wherein the aerosol-modifying agent further comprises an active agent, a flavorant, or a combination thereof.

8. The aerosol generating article of claim 1, wherein the active agent comprises a nicotine component, a cannabinoid, a terpene, caffeine, an amino acid, a vitamin, melatonin, a botanical extract, or a combination thereof.

9. The aerosol generating article of claim 1, further comprising a filter, a cooling element, or both a filter and a cooling element, optionally wherein the aerosol-modifying agent is present in the filter and/or the cooling element.

10. The aerosol generating article of claim 1, further comprising a mouthpiece having an upstream end and a downstream end, the mouthpiece comprising a hollow tubular element formed from filamentary tow at the downstream end of the mouthpiece.

11. The aerosol generating article of claim 10, wherein the mouthpiece comprises a body of material in the form of a cylinder having a longitudinal axis.

12. The aerosol generating article of claim 1, wherein the aerosol-modifying agent is retained within an aerosol-modifying agent release component.

13. The aerosol generating article of claim 12, wherein the aerosol-modifying agent release component is in the form of a coating, a capsule, a thread, a bead, or a paper.

14. The aerosol generating article of claim 12, wherein the aerosol-modifying agent release component is present in the form of one or more capsules comprising a shell encapsulating the aerosol-modifying agent, and optionally wherein the mouthpiece comprises a body of material in the form of a cylinder having a longitudinal axis, and wherein the one or more capsules are embedded within the body of material such that the one or more capsules are surrounded on all sides by the material forming the body of material.

15. The aerosol generating article of claim 14, wherein the shell comprises a barrier material comprising a gelling agent, a bulking agent, a filler, a buffer, a coloring agent, a plasticizer, or a combination thereof.

16. The aerosol generating article of claim 14, wherein the one or more capsules are spherical and have a diameter in a range from about 0.4 mm to about 3 mm.

17. The aerosol generating article of claim 1, comprising a wrapper which at least partially surrounds the article, and wherein the aerosol-modifying agent is absorbed or adsorbed in or on the wrapping material.

18. The aerosol generating article of claim 17, wherein the wrapper comprises ventilation apertures.

19. The aerosol generating article of claim 1, wherein the aerosol generating material is in the form of a rod, optionally wherein the rod has a length in a range from about 10 mm to about 100 mm.

20. The aerosol generating article of claim 1, wherein the rod has a cavity therein, and wherein an aerosol-modifying agent release component is present within the cavity in the form of one or more capsules comprising a shell encapsulating the aerosol-modifying agent.

21. The aerosol generating article of claim 1, wherein the aerosol generating material is a solid and optionally comprises a botanical material.

22. The aerosol generating article of claim 1, which is substantially free of a nicotine component.

23. The aerosol generating article of claim 1, which is substantially free of tobacco material.

24. An aerosol generating assembly comprising:
(i) an aerosol generating device; and
(ii) the aerosol generating article of claim 1, wherein the aerosol generating article and the aerosol generating device are arranged with respect to each other such that the aerosol generating material is heatable by the aerosol generating device.

25. The aerosol generating assembly of claim 24, wherein the aerosol generating material is in the form of a rod having a cavity therein, and wherein an aerosol-modifying agent release component is present within the cavity in the form of one or more capsules comprising a shell encapsulating the aerosol-modifying agent, and wherein the aerosol generating device comprises an aerosol generator adapted to enter the cavity in the rod and pierce the one or more capsules to release the aerosol-modifying agent.
